SEQUENCE LISTING (21 sequences)

SEQ ID NO: 1 - DNA, 16 bp, Artificial Sequence
  Synthetic oligonucleotide
ggcgcggtggctcacg

SEQ ID NO: 2 - DNA, 23 bp, Artificial Sequence
  Synthetic oligonucleotide
ttttttgagacggagtctcgctc

SEQ ID NO: 3 - DNA, 20 bp, Artificial Sequence
  Synthetic oligonucleotide
atggatgatttcgcagcttt

SEQ ID NO: 4 - DNA, 20 bp, Artificial Sequence
  Synthetic oligonucleotide
tggctttacaccaacgaaaa

SEQ ID NO: 5 - DNA, 20 bp, Artificial Sequence
  Synthetic oligonucleotide
tacagcggagcaactgaaga

SEQ ID NO: 6 - DNA, 20 bp, Artificial Sequence
  Synthetic oligonucleotide
ttgcagcacagagacaagaa

SEQ ID NO: 7 - DNA, 20 bp, Artificial Sequence
  Synthetic oligonucleotide
ccgggagagttttaccacaa

SEQ ID NO: 8 - DNA, 20 bp, Artificial Sequence
  Synthetic oligonucleotide
ccttccctagatgggacaca

SEQ ID NO: 9 - DNA, 20 bp, Artificial Sequence
  Synthetic oligonucleotide
ctgacctcacctgggacaat

SEQ ID NO: 10 - DNA, 20 bp, Artificial Sequence
  Synthetic oligonucleotide
cctccacaggtttcctggta

SEQ ID NO: 11 - DNA, 20 bp, Artificial Sequence
  Synthetic oligonucleotide
tttgggaccactgtctctcc

SEQ ID NO: 12 - DNA, 20 bp, Artificial Sequence
  Synthetic oligonucleotide
ctgaccattccccaggtcta

SEQ ID NO: 13 - DNA, 20 bp, Artificial Sequence
  Synthetic oligonucleotide
atggcttctcatcgtctgct

SEQ ID NO: 14 - DNA, 20 bp, Artificial Sequence
  Synthetic oligonucleotide
gctcctcattccttgggatt

SEQ ID NO: 15 - DNA, 20 bp, Artificial Sequence
  Synthetic oligonucleotide
gtgcctttatcacccatgct

SEQ ID NO: 16 - DNA, 20 bp, Artificial Sequence
  Synthetic oligonucleotide
tcttggatttgtgggctttc

SEQ ID NO: 17 - DNA, 1802 bp, Homo sapiens
  CDS (127)..(606)
gggcagctccaccctgggagggactgtggcccaggtactgcccgggtgctactttatgggcagcagctcagttgagttagagtctggaagacctcagaagacctcctgtcctatgaggccctccccatggctttagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaagaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 18 - protein, 159 aa, Homo sapiens
MALETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 19 - DNA, 2316 bp, Mus musculus
  CDS (36)..(572)
gtcgctagtctctattgccttgctgtggcctcgggatggaaatctgctggggaccctacagtcacctaatctctctccttctcatccttctgtttcattcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaatagtactgccgaggcctgtaataatcaccaactgcctgatcactctggccatcattggggcctgaggaacaacttttgcagggtgtatgtacagtagaaggagacagaagagttctgatgatagatctctgcctcagtctgttggctggcctaatccccatgatgattccagaataatcttgcaaattggatcatggcaggtgcttgttcaaagccctttcttgttgcctctgccatctgggtgaagtctagaccacttgcttggcctaggtgtcttctgctctaccacccaccctacccctgccacaaacacacactttttttgtttttgttttttccattgttctgcacttccacagtccagaccaatcaagtcacttgacaatatgccccaagtgactcccttaccctgttttataaacctgtgcctgtctatggagaaggttttaattctccttgttattcattttgggctttttgatgaaaccaccagggcatcacatatactaagcatgtgctctaccatcatgctatgcttccagctcaggggggcacttttaaggatctagaaaacagaaattaaggatctcatagttattttattaggccagccttattccatgtcggcaagaggtttcttgtggaaattatgtcctttctgagaggagctggggattagatgctcctgcatttgtgaaatggttataagcatagaaaaataggtggtaagctttccttctttccttattttgtgtgatgccttaaactgaaaagttaaaaattgatggattgtagcattcccataatctcccccttctttttttttcctttggaaatgtccaatagtctatattcctctgtcccgcccaaacaccatcttcactccaagcctaccacagatgcctgaagaagttcctcactatctgcaaatgtggctctcaggcccttcctgatgtgatgaatgaatctactaatcatttcttgaccattcattttatcacttctaaccttgaaacatgtggaagtagctatgttcctgactgtttcctctgccagacaatgaactctggagatcagggagcttcgtgtgtgtgtgtgtgtgtgtgtgtgtgtgtgtgtgtgtgtgtgcgtgcgcgcgcgcgtgcgcacgcacgtgcatgcacatgctatgtattgggtccctccaaggatgaaccctctctttggcttagaaggcactcagagaatatgtgttattcgtgctcacggaaagtttcttattcatccctgtgactttggctttattttacaataaaacactgaaaatgtccactttgttagttgtgaacatgagcccaggcctaaggtgctgggaaacagaaagggcgggagatttttctttattctatggctagaaaatagttacctcctctctgaaagtcttcttcctcatttctgggtaacagaatatcaaacaccttgcttataagttataaagtagtgttgtccaccatgaacccaccaagtaaaaacaacccaaatacctatcatggatgaataatcatgcaagtatcagatctgcactcaatgccacacaatgacaaagatagcaaatgagccacagacggctccacccaacccaatagatgaacacttggttcaaaatcactaaagctcaaatactcccaggtcaaacaccaggtaacaagttaatactcaacaaagggggaaaacaaatgttccactgaatcctgtgaccctgtgggcg

SEQ ID NO: 20 - protein, 178 aa, Mus musculus
MEICWGPYSHLISLLLILLFHSEAACRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 21 - protein, 153 aa, Artificial Sequence
  Synthetic polypeptide
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

(19) United States
(12) Patent Application Publication
(10) Pub. No.: US 2012/0045764 A1
(43) Pub. Date: Feb. 23, 2012

(54) METHOD OF EXPANDING HUMAN HEPATOCYTES IN VIVO

(76) Inventors: Markus Grompe, Portland, OR (US); Hongxiang Lan, Portland, OR (US)

(21) Appl. No.: 13/318,310

(22) PCT Filed: Apr. 30, 2010
(86) PCT No.: PCT/US2010/033210

§ 371 Date: Oct. 31, 2011

Related U.S. Application Data
(60) Provisional application No. 61/174,791, filed on May 1, 2009.
(60) Provisional application No. 61/296,774, filed on Jan. 20, 2010.

(51) Int. Cl. C12Q 1/68; G01N 33/566; G01N 21/64; C12N 5/071

(52) U.S. Cl. 435/6.11; 435/370; 435/7.1; 435/6.12

(57) ABSTRACT

Described herein is a method of expanding human hepatocytes in vivo using an immunodeficient mouse which is further deficient in fumarylacetoacetate hydrolase (Fah). The method comprises transplanting human hepatocytes into the immunodeficient and Fah-deficient mice, administering an IL-1R antagonist to the mouse and allowing the hepatocytes to expand. Alternatively, the method includes transplanting human hepatocytes into the immunodeficient and Fah-deficient mice, wherein the mouse is further deficient for IL-1R and allowing the hepatocytes to expand. The method also allows serial transplantation of the human hepatocytes into secondary, tertiary, quaternary or additional mice.

### CROSS REFERENCE TO RELATED APPLICATIONS

This application claims the benefit of U.S. Provisional Application No. 61/296,774, filed on Jan. 20, 2010, and U.S. Provisional Application No. 61/174,791, filed on May 1, 2009, which are herein incorporated by reference in their entirety.

### ACKNOWLEDGMENT OF GOVERNMENT SUPPORT

This invention was made with government support under grant number DK051592 awarded by the National Institutes of Health. The government has certain rights in the invention.

### FIELD

This disclosure is directed to a method for expanding hepatocytes (such as human hepatocytes), specifically to methods that utilize immunodeficient and Fah-deficient mice to expand hepatocytes.

### BACKGROUND

The liver is the principal site for the metabolism of xenobiotic compounds including medical drugs. Because many hepatic enzymes are species-specific, it is necessary to evaluate the metabolism of candidate pharmaceuticals using cultured primary human hepatocytes or their microsomal fraction (Brandon et al. Toxicol. Appl. Pharmacol. 189:233-246, 2003; Gomez-Lechon et al. Curr. Drug Metab. 4:292-312, 2003). While microsomal hepatocyte fractions can be used to elucidate some metabolic functions, other tests depend on living hepatocytes. Some compounds, for example, induce hepatic enzymes and thus their metabolism changes with time. To analyze enzyme induction, hepatocytes must be not only viable, but fully differentiated and functional.

For drug metabolism and other studies, hepatocytes are typically isolated from cadaveric organ donors and shipped to the location where testing will be performed. The condition (viability and state of differentiation) of hepatocytes from cadaveric sources is highly variable and many cell preparations are of marginal quality. The availability of high quality human hepatocytes is further hampered by the fact that they cannot be significantly expanded in tissue culture (Runge et al. Biochem. Biophys. Res. Commun. 274:1-3, 2000; Cascio S. M., Artif. Organs 25:529-538, 2001). After plating, the cells survive but do not divide. Hepatocytes from readily available mammalian species, such as the mouse, are not suitable for most drug testing studies because they have a different complement of metabolic enzymes and respond differently in induction studies. Immortal human liver cells (hepatomas) or fetal hepatoblasts are not an adequate replacement for fully differentiated adult cells. Human hepatocytes are also necessary for studies in the field of microbiology. Many human viruses, such as viruses which cause hepatitis, cannot replicate in any other cell type.

Given these limitations, methods of expanding primary human hepatocytes are highly desirable. Thus, provided herein is a robust system for expanding human hepatocytes.

### SUMMARY

Described herein are immunodeficient and Fah-deficient mice, which have utility for a variety of purposes, including for the expansion of hepatocytes from other species (particularly humans), and as animal models of human liver disease, including cirrhosis, fibrosis, hepatocellular carcinoma (HCC) and hepatic infection.

Provided herein is a method of expanding human hepatocytes in vivo. The method includes transplanting human hepatocytes into an immunodeficient and fumarylacetoacetate hydrolase (Fah)-deficient mouse, allowing the hepatocytes to expand, and optionally collecting the human hepatocytes. In several embodiments, to improve engraftment efficiency, the immunodeficient and Fah-deficient mouse is administered an IL-1R antagonist or the mouse is further deficient in IL-1R.

In some embodiments, the immunodeficient and Fah-deficient mice are administered an IL-1R antagonist during and/or after transplantation of the human hepatocytes.

In some embodiments, the immunodeficiency of the mouse is due to a genetic mutation, immunosuppression, or a combination thereof.

Also provided is a method for selecting an agent effective for the treatment of a human liver disease by administering a candidate agent to an immunodeficient and Fah-deficient mouse, wherein the mouse is further deficient for expression of IL-1R, or the mouse is administered an IL-1R antagonist, and assessing the effect of the candidate agent on the liver disease. An improvement in one or more signs or symptoms of the liver disease, indicates the candidate agent is effective for the treatment of the liver disease.

Further provided is a method for selecting an agent effective for the treatment of infection by a human hepatic pathogen by administering a candidate agent to an immunodeficient and Fah-deficient mouse transplanted with human hepatocytes, wherein the mouse is further deficient for expression of IL-1R, or the mouse is administered an IL-1R antagonist, and wherein the transplanted human hepatocytes are infected with the hepatic pathogen; and assessing the effect of the candidate agent on the hepatic infection.

A method is also provided for selecting an agent effective for the treatment of cirrhosis by administering a candidate agent to an immunodeficient and Fah-deficient mouse transplanted with human hepatocytes, and assessing the effect of the candidate agent on at least one diagnostic marker of cirrhosis in the mouse. In this method, the immunodeficient and Fah-deficient mouse is further deficient for expression of IL-1R, or the mouse is administered an IL-1R antagonist. Further provided is a method for selecting an agent effective for the treatment of HCC by administering a candidate agent to an immunodeficient and Fah-deficient mouse transplanted with human hepatocytes, wherein the immunodeficient and Fah-deficient mouse is further deficient for expression of IL-1R, or the mouse is administered an IL-1R antagonist, and assessing the effect of the candidate agent on HCC in the mouse.

A method of assessing the effect of an exogenous agent on human hepatocytes in vivo is also provided. In some embodiments, the method includes administering the exogenous agent to an immunodeficient and Fah-deficient mouse, wherein the immunodeficient and Fah-deficient mouse is further deficient for expression of IL-1R, or the mouse is administered an IL-1R antagonist; and measuring at least one marker of liver function in the mouse.

Further provided are methods of evaluating gene therapy protocols and vectors for the liver (including gene expression and gene knockdown vectors); drug metabolism, pharmacokinetics, efficacy, toxicology and safety; and human genetic liver diseases. Such methods can utilize immunodeficient and Fah-deficient mice transplanted with human hepatocytes, wherein the immunodeficient and Fah-deficient mouse is further deficient for expression of IL-1R, or the mouse is administered an IL-1R antagonist, or can utilize human hepatocytes that have been expanded in and collected from such mice.

The foregoing and other objects, features, and advantages of the invention will become more apparent from the following detailed description, which proceeds with reference to the accompanying figures.

### BRIEF DESCRIPTION OF THE FIGURES

FIG. 1A is a graph showing the relative weight of a triple mutant FRG mouse (#471) and two heterozygote littermates (#469, #470) following engraftment and repopulation by human hepatocytes. The FRG mouse maintained its weight 6 weeks after transplantation; however, the Il2rg gene heterozygote littermates lost weight continuously. NTBC was administered only in the first and the forth week, which is indicated by shading. FIG. 1B is a digital image of a gel showing PCR amplification products of human Alu sequence on genomic DNA from hepatocyte-recipient livers. Only FRG mice were positive. FIGS. 1C-1E are graphs showing FAH enzyme activity in wild-type (C), Fah^{-/-} (D) and humanized mouse liver (E). FAH substrate concentration declined in wild type mouse liver, but did not change with Fah^{-/-} mouse liver. Humanized mouse liver showed ample enzyme activity. FIG. 1F is a digital image of FAH immunostaining in a repopulated mouse liver showing more than 80% of hepatocytes are positive for FAH (indicated by dark staining and the large arrows). The small arrow demarks FAH negative cells. FIG. 1G is a digital image of H&E staining of the same liver section, which shows that human hepatocytes are less eosinophilic (indicated by the arrow). Original magnification ×200.

FIGS. 2A-2H are digital images of histological and immunohistochemical tissue sections from chimeric mice. FIG. 2A is a digital image showing FAH-positive human hepatocytes were integrated in mouse liver tissue and did not disturb recipient liver microstructure. FIG. 2B is a digital image showing that highly repopulated chimeric livers also retained normal structure. FIGS. 2C and 2D are digital images of H&E stains showing human hepatocyte clusters are less eosinophilic. FIG. 2E is a digital image showing serial sections stained for FAH. FIG. 2F is a digital image showing serial sections stained for HepPar. FIG. 2G is a digital image of a kidney section from a highly repopulated mouse showing no tubular or glomerular destruction even 4 months after transplantation. FIG. 2H is a digital image showing FAH positive human hepatocytes in the spleen. Original magnification ×100 (FIG. 2C), ×200 (FIGS. 2A, 2B, 2E, 2F and 2G), ×400 (FIGS. 2D and 2H).

FIG. 3A is a series of gel sections showing RT-PCR products from chimeric mouse liver. The human ALB, FAH, TAT, TF, TTR, and UGT1A1 genes were expressed in chimeric mice livers (#697 and #785). Human hepatocytes and mouse hepatocytes were used as positive and negative control respectively. FIG. 3B (normal plotting) and FIG. 3C (logarithmic plotting) are graphs showing blood human albumin concentration of primary hepatocyte recipients using ELISA. The threshold concentration of the system is approximately 0.005 μg/ml. FIG. 3D (normal plotting) and FIG. 3E (logarithmic plotting) are graphs showing human albumin concentration of secondary recipients. Logarithmic plotting shows the doubling time of albumin concentration is approximately one week.

FIG. 4A is a schematic showing the serial transplantation scheme starting with primary cells (dark box at far left). Dark boxes indicate repopulated serial recipients and white boxes indicate non-engrafted mice. Only ¼ of the primary recipients were repopulated but all 6 secondary recipients were engrafted. FIG. 4B is a digital image of a gel showing amplification products of Alu sequence PCR from serially transplanted recipient livers. FIGS. 4C-4E are digital images of hepatocytes analyzed by FAH immunocytochemistry demonstrating that more than 70% of cultured hepatocytes from a tertiary mouse were positive for FAH. FIGS. 4F-4H are digital images of tissue sections showing FAH immunohistochemistry of serially transplanted mice liver. Primary (F), secondary (G) and tertiary (H) recipient livers were repopulated by human hepatocytes.

FIGS. 5A-5C are digital images of anti-mouse albumin and anti-FAH immunocytochemistry of chimeric mouse hepatocytes. Most hepatocytes from chimeric liver were mouse albumin or FAH single positive. FIGS. 5D-5F are digital images of anti-human albumin and anti-FAH immunocytochemistry of chimeric mouse hepatocytes. Most hepatocytes were human albumin and FAH double positive. Original magnification ×100. FIGS. 5G-5L are graphs that show flow cytometric analysis of chimeric mouse hepatocytes. FITC-conjugated anti-HLA A,B,C and PE-conjugated anti-H-2 Kb were used. Shown are control human hepatocytes against HLA-A,B,C (G); control mouse hepatocytes against HLA-A,B,C (I); control human hepatocytes against H-2 Kb (H); control mouse hepatocytes against H-2 Kb (J); and hepatocytes from two highly chimeric mice (K and L), which were singly positive for either HLA or H-2 Kb.

FIGS. 6A and 6B are graphs showing metabolism of Ethoxyresorufin-O-deethylase (CYP1A1 dependent) (A) and conversion of testosterone to 6-beta-hydroxyltestosterone (CYP3A4 mediated) (B). Cultured hepatocytes from three mice with different levels of human hepatocyte repopulation (M790 10%; M697 30%; and M785 60%) were analyzed. FIG. 6C is a graph depicting mRNA levels of human specific genes relevant to drug metabolism, transport and conjugation, determined by quantitative RT-PCR. The ratios of human drug metabolism genes are typical of adult human hepatocytes.

FIGS. 7A-7H are graphs depicting basal gene expression levels of liver-specific genes and genes involved with drug metabolism in hepatocytes from three mice with different levels of human hepatocyte repopulation (M790 10%; M697 30%; and M785 60%). FIG. 7A is a bar graph of basal expression of liver-specific genes in the three samples, normalized to mouse actin mRNA. FIGS. 7B-7H are bar graphs of induction of mRNAs involved in drug metabolism in response to beta-naphthoflavone (BNF), phenobarbital (PB) and rifampicin (Rif), relative to induction in non-induced cultures. Shown are CYP3A4 (B); CYP2B6 (C); CAR (nuclear hormone receptor) (D); MDR1 (transporter) (E); MRP (F); BSEP (transporter) (G); and PXR (nuclear hormone receptor) (H). The induction of CYP3A4 by phenobarbital was even more striking than at the enzyme level.

FIG. 8 is a FACS plot of hepatocytes isolated from humanized FRG mice. Cells were co-stained with an anti-human CD46 and anti-mouse hepatocyte (HC) surface marker. The majority (>85%) of hepatocytes were human.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

The Sequence Listing is submitted as an ASCII text file, Annex C/St.25 text file, created on Apr. 16, 2010, 17 KB, which is incorporated by reference herein.

In the accompanying sequence listing:

SEQ ID NOs: 1 and 2 are the nucleic acid sequences of the PCR primers for amplifying human Alu sequences.

SEQ ID NO: 3 is the nucleic acid sequence of the human ALB forward RT-PCR primer.

SEQ ID NO: 4 is the nucleic acid sequence of the human ALB reverse RT-PCR primer.

SEQ ID NO: 5 is the nucleic acid sequence of the mouse Alb forward RT-PCR primer.

SEQ ID NO: 6 is the nucleic acid sequence of the mouse Alb reverse RT-PCR primer.

SEQ ID NO: 7 is the nucleic acid sequence of the human TAT forward RT-PCR primer.

SEQ ID NO: 8 is the nucleic acid sequence of the human TAT reverse RT-PCR primer.

SEQ ID NO: 9 is the nucleic acid sequence of the human TF forward RT-PCR primer.

SEQ ID NO: 10 is the nucleic acid sequence of the human TF reverse RT-PCR primer.

SEQ ID NO: 11 is the nucleic acid sequence of the human FAH forward RT-PCR primer.

SEQ ID NO: 12 is the nucleic acid sequence of the human FAH reverse RT-PCR primer.

SEQ ID NO: 13 is the nucleic acid sequence of the human TTR forward RT-PCR primer.

SEQ ID NO: 14 is the nucleic acid sequence of the human TTR reverse RT-PCR primer.

SEQ ID NO: 15 is the nucleic acid sequence of the human UGT1A1 forward RT-PCR primer.

SEQ ID NO: 16 is the nucleic acid sequence of the human UGT1A1 reverse RT-PCR primer.

SEQ ID NOs: 17 and 18 are the nucleic acid and amino acid sequences, respectively, of human IL-1RA (deposited under GenBank Accession No. NM_{—}000577.3 on Jan. 24, 2003).

SEQ ID NOs: 19 and 20 are the nucleic acid and amino acid sequences, respectively, of mouse IL-1RA (deposited under GenBank Accession No. NM_{—}001039701 on Apr. 6, 2007).

SEQ ID NO: 21 is the amino acid sequence of anakinra.

### DETAILED DESCRIPTION

### I. Abbreviations

AAV Adeno-associated virus

ALB Albumin

ALT Alanine aminotransferase

AST Aspartate aminotransferase

BNF Beta-naphthoflavone

CMV Cytomegalovirus

DAB Diaminobenzidine

ELISA Enzyme-linked immunosorbent assay

EROD Ethoxyresorufin-O-deethylase

ES Embryonic stem

FACS Fluorescence-activated cell sorting

FAH Fumarylacetoacetate hydrolase

FISH Fluorescence in situ hybridization

FITC Fluorescein isothiocyanate

FRG Fah^{-/-}/Rag2^{-/-}/Il2rg^{-/-} triple mutant mice

H&E Hematoxylin and eosin

HBV Hepatitis B virus

HCV Hepatitis C virus

HLA Human leukocyte antigen

HT1 Hereditary tyrosinemia type 1

IL-1 Interleukin-1

IL-1R Interleukin-1 receptor

IL-IRA Interleukin-1 receptor antagonist

IL-TRAP Interleukin-1 receptor accessory protein

IL-2Rγ Interleukin-2 receptor gamma

iPS Induced pluripotent stem

IPSC Induced pluripotency stem cells

MHC Major histocompatibility complex

mTOR Mammalian target of rapamycin

NOD Non-obese diabetic

NTBC 2-(2-nitro-4-trifluoro-methyl-benzoyl)-1,3 cyclohexanedione

PB Phenobarbital

PBS Phosphate-buffered saline

PCR Polymerase chain reaction

PE Phycoerythrin

PFU Plaque forming units

RAG Recombinase activating gene

Rif Rifampicin

RT-PCR Reverse transcription polymerase chain reaction

SA Succinylacetone

SCID Severe combined immunodeficiency

TAT Tyrosine aminotransferase

TF Transferrin

TTR Transthyretin

UGT1A1 UDP glucuronosyltransferase 1 family, polypeptide A1

uPA Urokinase plasminogen activator

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanations of specific terms are provided:

Administration: To provide or give a subject an agent, such as a therapeutic agent, by any effective route. Exemplary routes of administration include, but are not limited to, injection (such as subcutaneous, intramuscular, intradermal, intraperitoneal, and intravenous), oral, intraductal, sublingual, rectal, transdermal, intranasal, vaginal and inhalation routes.

Agent that inhibits or prevents the development of liver disease: A compound or composition that when administered to an FRG mouse, an F^{pm}RG mouse, or any other type of Fah-deficient mouse, prevents, delays or inhibits the development of liver disease in the mouse. Liver disease or liver dysfunction is characterized by any one of a number of signs or symptoms, including, but not limited to an alteration in liver histology (such as necrosis, inflammation, fibrosis, dysplasia or hepatic cancer), an alteration in levels of liver-specific enzymes and other proteins (such as aspartate aminotransferase, alanine aminotransferase, bilirubin, alkaline phosphatase and albumin) or generalized liver failure. In one embodiment, the agent that inhibits liver disease is 2-(2-nitro-4-trifluoro-methyl-benzoyl)-1,3 cyclohexanedione (NTBC).

Amniocyte: A cell found in the amniotic fluid surrounding an embryo.

Anakinra. An interleukin-1 (IL-1) receptor antagonist. Anakinra blocks the biologic activity of naturally occurring IL-1 by competitively inhibiting the binding of IL-1 to the IL-1 receptor, which is expressed in many tissues and organs. IL-1 is produced in response to inflammatory stimuli and mediates various physiologic responses, including inflammatory and immunologic reactions. Anakinra is a recombinant, non-glycosylated version of human IL-1RA (IL-1 receptor antagonist) prepared from cultures of genetically modified Escherichia coli. The anakinra protein is 153 amino acids and has a molecular weight of approximately 17.3 kD and differs from native human IL-1RA (SEQ ID NO: 18) in that it has a single methionine residue on its amino terminus (the amino acid sequence of anakinra is set forth herein as SEQ ID NO: 21). Anakinra is also known as KINERET™.

Antagonist: A compound (such as drug, protein or small molecule) that counteracts the effects of another compound. In some cases, an antagonist binds to a specific cellular receptor, but does not elicit a biological response.

Antibody: A protein (or protein complex) that includes one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as the myriad of immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is generally a tetramer. Each tetramer is composed of two identical pairs of polypeptide chains, each pair having one “light” (about 25 kDa) and one “heavy” (about 50-70 kDa) chain. The N-terminus of each chain defines a variable region of about 100 to 110 or more amino acids primarily responsible for antigen recognition. The terms “variable light chain” (V_{L}) and “variable heavy chain” (V_{H}) refer, respectively, to these light and heavy chains.

As used herein, the term “antibodies” includes intact immunoglobulins as well as a number of well-characterized fragments. For instance, Fabs, Fvs, and single-chain Fvs (scFvs) that bind to target protein (or epitope within a protein or fusion protein) would also be specific binding agents for that protein (or epitope). These antibody fragments are defined as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable region of the light chain and the variable region of the heavy chain expressed as two chains; and (6) single chain antibody, a genetically engineered molecule containing the variable region of the light chain, the variable region of the heavy chain, linked by a suitable polypeptide linker as a genetically fused single chain molecule. Methods of making these fragments are routine (see, for example, Harlow and Lane, Using Antibodies: A Laboratory Manual, CSHL, New York, 1999).

Antibodies for use in the methods of this disclosure can be monoclonal or polyclonal. Merely by way of example, monoclonal antibodies can be prepared from murine hybridomas according to the classical method of Kohler and Milstein (Nature 256:495-497, 1975) or derivative methods thereof. Detailed procedures for monoclonal antibody production are described in Harlow and Lane, Using Antibodies: A Laboratory Manual, CSHL, New York, 1999.

Antigen: A compound, composition, or substance that can stimulate the production of antibodies or a T-cell response in an animal, including compositions that are injected or absorbed into an animal. An antigen reacts with the products of specific humoral or cellular immunity. A “pathogen-specific antigen” is an antigen, such as a protein, expressed by a pathogen, such as a virus, bacteria or parasite that elicits an immune response in a subject.

Azathioprine: An immunosuppressant that is a purine synthesis inhibitor, inhibiting the proliferation of cells, especially leukocytes. This immunosuppressant is often used in the treatment of autoimmune diseases or organ transplant rejection. It is a pro-drug, converted in the body to the active metabolites 6-mercaptopurine (6-MP) and 6-thioinosinic acid. Azathioprine is produced by a number of generic manufacturers and as branded names (Azasan™ by Salix; Imuran™ by GlaxoSmithKline; Azamun™; and Imurel™)

B cell: A type of lymphocyte that plays a large role in the humoral immune response. The principal function of B cells is to make antibodies against soluble antigens. B cells are an essential component of the adaptive immune system.

Biological sample or sample: A sample obtained from cells, tissue or bodily fluid of a subject, such as peripheral blood, serum, plasma, cerebrospinal fluid, bone marrow, urine, saliva, tissue biopsy, surgical specimen, and autopsy material.

Cirrhosis: Refers to a group of chronic liver diseases characterized by loss of the normal microscopic lobular architecture and regenerative replacement of necrotic parenchymal tissue with fibrous bands of connective tissue that eventually constrict and partition the organ into irregular nodules. Cirrhosis has a lengthy latent period, usually followed by sudden abdominal pain and swelling with hematemesis, dependent edema, or jaundice. In advanced stages there may be ascites, pronounced jaundice, portal hypertension, varicose veins and central nervous system disorders that may end in hepatic coma.

Collecting: As used herein, “collecting” expanded human hepatocytes refers to the process of removing the expanded hepatocytes from a mouse that has been injected with isolated human hepatocytes (also referred to as a recipient mouse). Collecting optionally includes separating the hepatocytes from other cell types. In one embodiment, the expanded human hepatocytes are collected from the liver of a Fah-deficient mouse. In some examples, the expanded human hepatocytes are collected from the liver of an FRG mouse or an F^{pm}RG mouse.

Common-γ chain of the interleukin receptor (Il2rg): A gene encoding the common gamma chain of interleukin receptors. Il2rg is a component of the receptors for a number of interleukins, including IL-2, IL-4, IL-7 and IL-15 (Di Santo et al. Proc. Natl. Acad. Sci. U.S.A. 92:377-381, 1995). Animals deficient in Il2rg exhibit a reduction in B cells and T cells and lack natural killer cells. Il2rg is also known as interleukin-2 receptor gamma chain.

Cryopreserved: As used herein, “cryopreserved” refers to a cell or tissue that has been preserved or maintained by cooling to low sub-zero temperatures, such as 77 K or −196° C. (the boiling point of liquid nitrogen). At these low temperatures, any biological activity, including the biochemical reactions that would lead to cell death, is effectively stopped.

Cyclosporin A: An immunosuppressant compound that is a non-ribosomal cyclic peptide of 11 amino acids produced by the soil fungus Beauveria nivea. Cyclosporin A is used for the prophylaxis of graft rejection in organ and tissue transplantation. Cyclosporin A is also known as cyclosporine and ciclosporin.

Decreased liver function: An abnormal change in any one of a number of parameters that measure the health or function of the liver. Decreased liver function is also referred to herein as “liver dysfunction.” Liver function can be evaluated by any one of a number of means well known in the art, such as, but not limited to, examination of liver histology and measurement of liver enzymes or other proteins. For example, liver dysfunction can be indicated by necrosis, inflammation, fibrosis, oxidative damage or dysplasia of the liver. In some instances, liver dysfunction is indicated by hepatic cancer, such as hepatocellular carcinoma. Examples of liver enzymes and proteins that can be tested to evaluate liver dysfunction include, but are not limited to, alanine aminotransferase (ALT), aspartate aminotransferase (AST), bilirubin, alkaline phosphatase and albumin. Liver dysfunction also can result in generalized liver failure. Procedures for testing liver function are well known in the art, such as those taught by Grompe et al. (Genes Dev. 7:2298-2307, 1993) and Manning et al. (Proc. Natl. Acad. Sci. U.S.A. 96:11928-11933, 1999).

Deficient: As used herein, “Fah-deficient” or “deficient in Fah” refers to an animal, such as a mouse, comprising a mutation in Fah, which results in a substantial decrease in, or the absence of, Fah mRNA expression and/or functional FAH protein. As used herein, the term “loss of expression” of functional FAH protein does not refer to only a complete loss of expression, but also includes a substantial decrease in expression of functional FAH protein, such as a decrease of about 80%, about 90%, about 95% or about 99%. In one embodiment, the Fah-deficient animal comprises homozygous disruptions, such as homozygous deletions, in the Fah gene. A disruption includes, for example, an insertion, deletion, one or more point mutations, or any combination thereof. As one example, the homozygous deletion is in exon 5 of Fah. In another embodiment, the Fah-deficient animal comprises one or more point mutations in the Fah gene. Examples of suitable Fah point mutations are known in the art (see, for example, Aponte et al. Proc. Natl. Acad. Sci. U.S.A. 98(2):641-645, 2001). Similarly, “IL-1R-deficient” or “deficient in IL-1R” refers to an animal, such as a mouse, comprising a mutation in IL-1R, which results in a substantial decrease in, or the absence of, IL-1R mRNA expression and/or functional IL-1R protein. IL-1R knockout mice have been previously described (see, for example, Norman et al., Ann. Surg. 223(2):163-169, 1996; Glaccum et al., J. Immunol. 159:3364-3371, 1997) and are commercially available, such as from The Jackson Laboratory (Bar Harbor, Me.). In addition, Rag1-deficient, Rag2-deficient, and Il2rg-deficient refer to animals comprising a mutation in Rag1, Rag2 and Il2rg, respectively, resulting in a substantial decrease in or absence of mRNA expression or production of functional protein. Rag1, Rag2 and Il2rg knockout mice have been previously described and are commercially available.

Deplete: To reduce or remove. As used herein, “macrophage depletion” refers to the process of eliminating, removing, reducing or killing macrophages in an animal. An animal that has been depleted of macrophages is not necessarily completely devoid of macrophages but at least exhibits a reduction in the number or activity of macrophages. In one embodiment, macrophage depletion results in at least a 10%, at least a 25%, at least a 50%, at least a 75%, at least a 90% or a 100% reduction in functional macrophages.

Disruption: As used herein, a “disruption” in a gene refers to any insertion, deletion or point mutation, or any combination thereof. In some embodiments, the disruption leads to a partial or complete loss of expression of mRNA and/or functional protein.

Embryonic stem (ES) cells: Pluripotent cells isolated from the inner cell mass of the developing blastocyst. ES cells are pluripotent cells, meaning that they can generate all of the cells present in the body (bone, muscle, brain cells, etc.). Methods for producing murine ES cells can be found in U.S. Pat. No. 5,670,372. Methods for producing human ES cells can be found in U.S. Pat. No. 6,090,622, PCT Publication No. WO 00/70021 and PCT Publication No. WO 00/27995. Also contemplated herein are induced pluripotent stem cells (iPS cells), which are a type of pluripotent stem cell artificially derived from a non-pluripotent cell (such as an adult somatic cell) by inducing expression of certain genes, such as OCT3/4, SOX2, NANOG, LIN28, Klf4, and/or c-Myc (Yu et al., Science 318(5858):1917-1920, 2007; Takahashi et al., Cell 131(5):861-872, 2007). Thus far, iPS cells from mouse (Okita et al., Nature 448(7151):313-317, 2007), human (Yu et al., Science 318(5858):1917-1920, 2007; Takahashi et al., Cell 131(5):861-872, 2007), rat (Li et al., Cell Stem Cell 4(1):16-19, 2009), monkey (Liu et al., Cell Stem Cell 3(6):587-590, 2008) and pig (Esteban et al., J. Biol. Chem. Epub Apr. 21, 2009) have been reported.

Engraft: To implant cells or tissues in an animal. As used herein, engraftment of human hepatocytes in a recipient mouse refers to the process of human hepatocytes becoming implanted in the recipient mouse following injection. Engrafted human hepatocytes are capable of expansion in the recipient mouse. As described herein, “significant engraftment” refers to a recipient mouse wherein at least about 1% of the hepatocytes in the liver are human. A “highly engrafted” mouse is one having a liver wherein at least about 60% of the hepatocytes are human. However, engraftment efficiency can be higher, such as at least about 70%, at least about 80%, at least about 90% or at least about 95% of the hepatocytes in the mouse liver are human hepatocytes.

Expand: To increase in quantity. As used herein, “expanding” human hepatocytes refers to the process of allowing cell division to occur such that the number of human hepatocytes increases. As described herein, human hepatocytes are allowed to expand in a recipient mouse for at least about four weeks, at least about six weeks, at least about 8 weeks, at least about 12 weeks, at least about 16 weeks, at least about 20 weeks, at least about 24 weeks or at least about 28 weeks. In one embodiment, the human hepatocytes are allowed to expand for up to about 6 months. In other embodiments, the human hepatocytes are allowed to expand for up to about 8, about 10 or about 12 months. The number of human hepatocytes resulting from expansion can vary. In one embodiment, expansion results in at least 10 million, at least 20 million, at least 30 million, at least 40 million or at least 50 million hepatocytes. Assuming one million hepatocytes are initially injected, and approximately 10% engraft, hepatocyte expansion can range from about 10-fold to about 500-fold. In some embodiments, expansion of human hepatocytes in a recipient mouse results in an increase of at least 10-fold, at least 50-fold, at least 100-fold, at least 150-fold, at least 200-fold, at least 250-fold, at least 300-fold, at least 400-fold, at least 500-fold or at least 1000-fold.

FK506: FK506, also known as tacrolimus or fujimycin, is an immunosuppressant drug. FK506a 23-membered macrolide lactone first discovered in the fermentation broth of a Japanese soil sample that contained the bacteria Streptomyces tsukubaensis. This compound is often used after allogeneic organ transplant to reduce the activity of the patient's immune system and lower the risk of organ rejection. FK506 reduces T-cell and interleukin-2 activity. It is also used in a topical preparation in the treatment of severe atopic dermatitis (eczema), severe refractory uveitis after bone marrow transplants, and the skin condition vitiligo.

Fludarabine: A purine analog that inhibits DNA synthesis. Fludarabine is often used as a chemotherapeutic drug for the treatment of various hematologic malignancies.

FRG mouse: A mutant mouse having homozygous deletions in the fumarylacetoacetate hydrolase (Fah), recombinase activating gene 2 (Rag2) and common-γ chain of the interleukin receptor (Il2rg) genes. Also referred to herein as Fah^{-/-}/Rag2^{-/-}/Il2rg^{-/-}. As used herein, homozygous deletions in the Fah, Rag2 and Il2rg genes indicates no functional FAH, RAG-2 and IL-2Rγ protein is expressed in mice comprising the mutations.

FP^{pm}RG mouse: A mutant mouse having homozygous deletions in the recombinase activating gene 2 (Rag2) and common-γ chain of the interleukin receptor (Il2rg) genes, and homozygous point mutations in the fumarylacetoacetate hydrolase (Fah). The point mutation in the Fah gene of F^{pm}RG mice results in missplicing and loss of exon 7 in the mRNA (Aponte et al., Proc. Natl. Acad. Sci. USA 98:641-645, 2001). Also referred to herein as Fah^{pm}/Rag2^{-/-}/Il2rg^{-/-}. As used herein, homozygous deletions in the Rag2 and Il2rg genes indicates no functional RAG-2 and IL-2Rγ protein is expressed in mice comprising the mutations. In addition, mice having homozygous point mutations in the Fah gene do not express functional FAH protein.

Fumarylacetoacetate hydrolase (FAH): A metabolic enzyme that catalyzes the last step of tyrosine catabolism. Mice having a homozygous deletion of the Fah gene exhibit altered liver mRNA expression and severe liver dysfunction (Grompe et al. Genes Dev. 7:2298-2307, 1993). Point mutations in the Fah gene have also been shown to cause hepatic failure and postnatal lethality (Aponte et al. Proc. Natl. Acad. Sci. U.S.A. 98(2):641-645, 2001). Humans deficient for Fah develop the liver disease hereditary tyrosinemia type 1 (HT1) and develop liver failure. Fah deficiency leads to accumulation of fumarylacetoacetate, a potent oxidizing agent and this ultimately leads to cell death of hepatocytes deficient for Fah. Thus, Fah-deficient animals can be repopulated with hepatocytes from other species, including humans.

Gradually reduced: As used herein, “gradually reducing” the dose of NTBC refers to the process of decreasing the dose of NTBC administered to Fah-deficient mice over time, such as over the course of several days. In one embodiment, the NTBC dose is gradually reduced over about a six day period, wherein the dose is decreased at about one or two day intervals such that after about one week, NTBC is no longer administered. The gradual reduction in NTBC can be performed over a shorter or longer period of time and the intervals of time between decreases in dose can also be shorter or longer.

Hepatic pathogen: Refers to any pathogen, such as a bacterial, viral or parasitic pathogen, that infects cells of the liver. In some embodiments, the hepatic pathogen is a “hepatotropic virus” (a virus that targets the liver), such as HBV or HCV.

Hepatocellular carcinoma (HCC): HCC is a primary malignancy of the liver typically occurring in patients with inflammatory livers resulting from viral hepatitis, liver toxins or hepatic cirrhosis.

Hepatocyte: A type of cell that makes up 70-80% of the cytoplasmic mass of the liver. Hepatocytes are involved in protein synthesis, protein storage and transformation of carbohydrates, synthesis of cholesterol, bile salts and phospholipids, and detoxification, modification and excretion of exogenous and endogenous substances. The hepatocyte also initiates the formation and secretion of bile. Hepatocytes manufacture serum albumin, fibrinogen and the prothrombin group of clotting factors and are the main site for the synthesis of lipoproteins, ceruloplasmin, transferrin, complement and glycoproteins. In addition, hepatocytes have the ability to metabolize, detoxify, and inactivate exogenous compounds such as drugs and insecticides, and endogenous compounds such as steroids.

Hereditary tyrosinemia type 1 (HT1): Tyrosinemia is an error of metabolism, usually inborn, in which the body cannot effectively break down the amino acid tyrosine. HT1 is the most severe form of this disorder and is caused by a shortage of the enzyme fumarylacetoacetate hydrolase (FAH) encoded by the gene Fah found on human chromosome number 15. FAH is the last in a series of five enzymes needed to break down tyrosine. Symptoms of HT1 usually appear in the first few months of life and include failure to gain weight and grow at the expected rate (failure to thrive), diarrhea, vomiting, yellowing of the skin and whites of the eyes (jaundice), cabbage-like odor, and increased tendency to bleed (particularly nosebleeds). HT1 can lead to liver and kidney failure, problems affecting the nervous system, and an increased risk of liver cancer.

Heterozygous: Having dissimilar alleles at corresponding chromosomal loci. For example, an animal heterozygous for a particular gene mutation has the mutation in one allele of the gene but not the other.

Homozygous: Having identical alleles at one or more loci. As used herein, “homozygous for disruptions” refers to an organism having identical disruptions (such as an insertion, deletion or point mutation) of both alleles of a gene.

Immunodeficient: Lacking in at least one essential function of the immune system. As used herein, and “immunodeficient” mouse is one lacking specific components of the immune system or lacking function of specific components of the immune system. In one embodiment, an immunodeficient mouse lacks functional B cells, T cells and/or NK cells. In another embodiment, an immunodeficient mouse further lacks macrophages. In some embodiments, an “immunodeficient mouse” comprises one or more of the following genetic alterations: Rag1^{-/-}, Rag2^{-/-}, Il2rg^{-/-}, SCID, NOD and nude. Immunodeficient mouse strains are well known in the art and are commercially available, such as from The Jackson Laboratory (Bar Harbor, Me.) or Taconic (Hudson, N.Y.). In some embodiments, an immunodeficient mouse is a mouse that has been administered one or more immunosuppressants.

Immunosuppressant: Any compound that decreases the function or activity of one or more aspects of the immune system, such as a component of the humoral or cellular immune system or the complement system. In particular embodiments of the disclosure, the immunosuppressant is FK506, cyclosporin A, fludarabine, mycophenolate, prednisone, rapamycin or azathioprine, or combinations thereof.

Known immunosuppressants include, but are not limited to: (1) antimetabolites, such as purine synthesis inhibitors (e.g., azathioprine and mycophenolic acid), pyrimidine synthesis inhibitors (e.g., leflunomide and teriflunomide) and antifolates (e.g., methotrexate); (2) macrolides, such as FK506, cyclosporine A and pimecrolimus; (3) TNF-α inhibitors, such as thalidomide and lenalidomide; (4) IL-1 receptor antagonists, such as anakinra; (5) mammalian target of rapamycin (mTOR) inhibitors, such as rapamycin (sirolimus), deforolimus, everolimus, temsirolimus, zotarolimus and biolimus A9; (6) corticosteroids, such as prednisone; and (7) antibodies to any one of a number of cellular or serum targets.

Exemplary cellular targets and their respective inhibitor compounds include, but are not limited to complement component 5 (e.g., eculizumab); tumor necrosis factors (TNFs) (e.g., infliximab, adalimumab, certolizumab pegol, afelimomab and golimumab); IL-5 (e.g., mepolizumab); IgE (e.g., omalizumab); BAYX (e.g., nerelimomab); interferon (e.g., faralimomab); IL-6 (e.g., elsilimomab); IL-12 and IL-13 (e.g., lebrikizumab and ustekinumab); CD3 (e.g., muromonab-CD3, otelixizumab, teplizumab, visilizumab); CD4 (e.g., clenoliximab, keliximab and zanolimumab); CD11a (e.g., efalizumab); CD18 (e.g., erlizumab); CD20 (e.g., afutuzumab, ocrelizumab, pascolizumab); CD23 (e.g., lumiliximab); CD40 (e.g., teneliximab, toralizumab); CD62L/L-selectin (e.g., aselizumab); CD80 (e.g., galiximab); CD147/basigin (e.g., gavilimomab); CD154 (e.g., ruplizumab); BLyS (e.g., Belimumab); CTLA-4 (e.g., ipilimumab, tremelimumab); CAT (e.g., bertilimumab, lerdelimumab, metelimumab); integrin (e.g., natalizumab); IL-6 receptor (e.g., Tocilizumab); LFA-1 (e.g., odulimomab); and IL-2 receptor/CD25 (e.g., basiliximab, daclizumab, inolimomab).

Other immunsuppressive agents include zolimomab aritox, atorolimumab, cedelizumab, dorlixizumab, fontolizumab, gantenerumab, gomiliximab, maslimomab, morolimumab, pexelizumab, reslizumab, rovelizumab, siplizumab, talizumab, telimomab aritox, vapaliximab, vepalimomab, anti-thymocyte globulin, anti-lymphocyte globulin; CTLA-4 inhibitors (e.g., abatacept, belatacept); aflibercept; alefacept; rilonacept; and TNF inhibitors (e.g., etanercept).

Immunosuppression: Refers to the act of reducing the activity or function of the immune system. Immunosuppression can be achieved by administration of an immunosuppressant compound or can be the effect of a disease or disorder (for example, immunosuppression that results from HIV infection or due to a genetic defect).

Interleukin-1 (IL-1): The term “IL-1” includes both IL-1α and IL-1β. IL-1α is a pleiotropic cytokine involved in various immune responses, inflammatory processes, and hematopoiesis. IL-1α is produced by monocytes and macrophages as a proprotein, which is proteolytically processed and released in response to cell injury, and thus induces apoptosis. IL-1β is produced by activated macrophages as a proprotein, which is proteolytically processed to its active form by caspase 1. IL-1β is an important mediator of the inflammatory response, and is involved in a variety of cellular activities, including cell proliferation, differentiation and apoptosis.

Induced pluripotency stem cells (IPSC): A type of pluripotent stem cell artificially derived from a non-pluripotent cell, typically an adult somatic cell, by inducing expression of certain genes. IPSCs can be derived from any organism, such as a mammal. In some embodiments, IPSCs are produced from mice, rats, rabbits, guinea pigs, goats, pigs, cows, non-human primates or humans. Human derived IPSCs are exemplary.

IPSCs are similar to ES cells in many respects, such as the expression of certain stem cell genes and proteins, chromatin methylation patterns, doubling time, embryoid body formation, teratoma formation, viable chimera formation, and potency and differentiability. Methods for producing IPSCs are known in the art. For example, IPSCs are typically derived by transfection of certain stem cell-associated genes (such as Oct-3/4 (Pouf51) and Sox2) into non-pluripotent cells, such as adult fibroblasts. Transfection can be achieved through viral vectors, such as retroviruses, lentiviruses, or adenoviruses. For example, cells can be transfected with Oct3/4, Sox2, Klf4, and c-Myc using a retroviral system or with OCT4, SOX2, NANOG, and LIN28 using a lentiviral system. After 3-4 weeks, small numbers of transfected cells begin to become morphologically and biochemically similar to pluripotent stem cells, and are typically isolated through morphological selection, doubling time, or through a reporter gene and antibiotic selection. In one example, IPSCs from adult human cells are generated by the method of Yu et al. (Science 318(5854):1224, 2007) or Takahashi et al. (Cell 131(5):861-72, 2007). IPSCs are also known as iPS cells.

Infectious load: Refers to the quantity of a particular pathogen in a subject or in a sample from the subject. Infectious load can be measured using any one of a number of methods known in the art. The selected method will vary depending on the type of pathogen to be detected and the reagents available to detect the pathogen. Infectious load can also be measured, for example, by determining the titer of the pathogen, the method for which will vary depending on the pathogen to be detected. For example, the titer of some viruses can be quantified by performing a plaque assay. In some examples, infectious load is measured by quantifying the amount of a pathogen-specific antigen in a sample. In other examples, infectious load is measured by quantifying the amount of a pathogen-specific nucleic acid molecule in a sample. Quantifying encompasses determining a numerical value or can be a relative value.

Interleukin-1 receptor (IL-1R): A cytokine receptor that belongs to the interleukin 1 receptor family. This protein is a receptor for IL-1α, IL-1β, and interleukin-1 receptor antagonist (IL-1RA). It is an important mediator involved in many cytokine induced immune and inflammatory responses. The term “IL-1R” generally includes both IL-1R type I and IL-1R type II. In the context of the present disclosure, “IL-1R” refers to IL-1R type I.

IL-1R antagonist (IL-IRA): A molecule that binds to IL-1R or IL-1R accessory protein and inhibits the activation mediated by the IL-1R. IL-1R antagonist (IL-1RA) also is the name of a particular protein, which is a member of the IL-1 cytokine family that binds to the same receptor on the cell surface as IL-1. The IL-1RA protein inhibits the activities of IL-1α and IL-1β, and modulates a variety of IL-1 related immune and inflammatory responses. IL-1RA is also known as IRAP, IL1RA, IL-1ra3 and IL1RN.

Interleukin-1 receptor accessory protein (IL-1RAP): This gene encodes an IL-1 receptor accessory protein. IL-1 induces synthesis of acute phase and proinflammatory proteins during infection, tissue damage, or stress, by forming a complex at the cell membrane with IL-1R and IL-1RAP. Alternative splicing of IL-1RAP results in two transcript variants encoding two different isoforms, one membrane-bound and one soluble. IL-1RAP is also known as IL1R3, IL-1RAcP and IL1RAP.

Isolated: An “isolated” biological component, such as a nucleic acid, protein (including antibodies) or organelle, has been substantially separated or purified away from other biological components in the environment (such as a cell) in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

An “isolated hepatocyte” refers to a hepatocyte that has been obtained from a particular source, such as an organ donor. In some embodiments, an “isolated hepatocyte” is a hepatocyte that has been removed from the body of a donor. In some embodiments, “isolated hepatocytes” are hepatocytes in suspension or hepatocytes contained within a piece of tissue. In particular examples, isolated hepatocytes are those that are substantially separated or purified away from other cell types, or purified away from other types of tissue, such as adipose tissue or fibrotic tissue.

Macrophage: A cell within the tissues that originates from specific white blood cells called monocytes. Monocytes and macrophages are phagocytes, acting in both nonspecific defense (or innate immunity) as well as specific defense (or cell-mediated immunity) of vertebrate animals. Their role is to phagocytize (engulf and then digest) cellular debris and pathogens either as stationary or mobile cells, and to stimulate lymphocytes and other immune cells to respond to the pathogen.

Mammalian target of rapamycin (mTOR) inhibitor: A molecule that inhibits expression or activity of mTOR. mTOR inhibitors include, but are not limited to small molecule, antibody, peptide and nucleic acid inhibitors. For example, an mTOR inhibitor can be a molecule that inhibits the kinase activity of mTOR or inhibits binding of mTOR to a ligand. Inhibitors of mTOR also include molecules that down-regulate expression of mTOR. A number of mTOR inhibitors are known in the art, including rapamycin (sirolimus).

Mycophenolate: An immunosuppressant typically used to prevent rejection of allogeneic transplants. This drug is generally administered orally or intravenously. Mycophenolate is derived from the fungus Penicillium stoloniferum. Mycophenolate mofetil, the pro-drug form, is metabolized in the liver to the active moiety mycophenolic acid. It inhibits inosine monophosphate dehydrogenase, the enzyme that controls the rate of synthesis of guanine monophosphate in the de novo pathway of purine synthesis used in the proliferation of B and T lymphocytes. Mycophenolic acid is commonly marketed under the trade names CellCept™ (mycophenolate mofetil; Roche) and Myfortic™ (mycophenolate sodium; Novartis).

Natural Killer (NK) cell: A form of cytotoxic lymphocyte which constitute a major component of the innate immune system. NK cells play a major role in the host-rejection of both tumors and virally infected cells.

Non-obese diabetic (NOD) mouse: Refers to a mouse that is genetically predisposed to the spontaneous development of autoimmune insulin dependent diabetes mellitus (IDDM). The susceptibility to IDDM is polygenic and environment exerts a strong effect on gene penetrances. The NOD strain was developed at Shionogi Research Laboratories in Aburahi, Japan (Makino et al., “Establishment of the non-obese diabetic (NOD) mouse,” In Current Topics in Clinical and Experimental Aspects of Diabetes Mellitus. Elsevier, Amsterdam, pages 25-32, 1985; Makino et al., Exp. Anim. 29:1-8, 1980).

NTBC (2-nitro-4-trifluoro-methyl-benzoyl)-1,3 cyclohexanedione): An inhibitor of 4-hydroxy-phenylpyruvate dioxygenase (HPPD). HPPD catalyzes the conversion of 4-hydroxyphenylpyruvate to homogentisate, the second step in tyrosine catabolism. Treatment with NTBC blocks the tyrosine catabolism pathway at this step and prevents the accumulation of succinylacetone, a pathognomonic metabolite that accumulates in Fah-deficient humans and animals.

Nude mouse: Refers to a mouse strain with a genetic mutation that causes a deteriorated or absent thymus, resulting in an inhibited immune system due to a greatly reduced number of T cells. The phenotypic appearance of the mouse is a lack of body hair. Nude mice have a spontaneous deletion in the forkhead box N1 (Foxn1) gene.

Prednisone: A synthetic corticosteroid that is an effective immunosuppressant. It is often used to treat certain inflammatory diseases, autoimmune diseases and cancers as well as treat or prevent organ transplant rejection. Prednisone is usually taken orally but can be delivered by intramuscular injection or intravenous injection. It is a prodrug that is converted by the liver into prednisolone, which is the active drug and also a steroid.

Rapamycin: A compound with known immunosuppressive and anti-proliferative properties. Rapamycin, also known as sirolimus, is a macrolide that was first discovered as a product of the bacterium Streptomyces hygroscopicus. Rapamycin binds and inhibits the activity of mTOR.

Recipient: As used herein, a “recipient mouse” is a mouse that has been injected with the isolated human hepatocytes described herein. Typically, a portion (the percentage can vary) of the human hepatocytes engraft in the recipient mouse. In one embodiment, the recipient mouse is an immunodeficient mouse which is further deficient in Fah. In another embodiment, the recipient mouse is a Rag2^{-/-}/Il2rg^{-/-} mouse which is further deficient in Fah. In another embodiment, the recipient mouse is an FRG mouse. In another embodiment, the recipient mouse is an F^{pm}RG mouse. In other embodiments, the recipient mouse is an FRG mouse treated with an IL-1R antagonist or an FRG mouse that is further deficient in IL-1R.

Recombinase activating gene 1 (Rag1): A gene involved in activation of immunoglobulin V(D)J recombination. The RAG1 protein is involved in recognition of the DNA substrate, but stable binding and cleavage activity also requires RAG2.

Recombinase activating gene 2 (Rag2): A gene involved in recombination of immunoglobulin and T cell receptor loci. Animals deficient in the Rag2 gene are unable to undergo V(D)J recombination, resulting in a complete loss of functional T cells and B cells (Shinkai et al., Cell 68:855-867, 1992).

Serial transplantation: The process for expanding human hepatocytes in vivo in which hepatocytes expanded in a first mouse are collected and transplanted, such as by injection, into a secondary mouse for further expansion. Serial transplantation can further include tertiary, quaternary or additional mice (Overturf et al., Am. J. Pathol. 151: 1078-9107, 1997).

Severe combined immunodeficiency (SCID) mouse: Refers to a strain of mice that is unable to undergo V(D)J recombination and therefore lack functional T cells and B cells. SCID mice also have an impaired ability to activate some components of the complement system. SCID mice are homozygous for the Prkdc^{scid} mutation.

Stem cell: A cell having the unique capacity to produce unaltered daughter cells (self-renewal; cell division produces at least one daughter cell that is identical to the parent cell) and to give rise to specialized cell types (potency). Stem cells include, but are not limited to, embryonic stem (ES) cells, embryonic germ (EG) cells, germline stem (GS) cells, human mesenchymal stem cells (hMSCs), adipose tissue-derived stem cells (ADSCs), multipotent adult progenitor cells (MAPCs), multipotent adult germline stem cells (maGSCs) and unrestricted somatic stem cell (USSCs). The role of stem cells in vivo is to replace cells that are destroyed during the normal life of an animal. Generally, stem cells can divide without limit. After division, the stem cell may remain as a stem cell, become a precursor cell, or proceed to terminal differentiation. A precursor cell is a cell that can generate a fully differentiated functional cell of at least one given cell type. Generally, precursor cells can divide. After division, a precursor cell can remain a precursor cell, or may proceed to terminal differentiation. In one embodiment, the stem cells give rise to hepatocytes.

T cell: A type of white blood cell, or lymphocyte, that plays a central role in cell-mediated immunity. T cells are distinguished from other types of lymphocytes, such as B cells and NK cells, by the presence of a special receptor on their cell surface that is called the T cell receptor (TCR). The thymus is generally believed to be the principal organ for T cell development.

Therapeutic agent: A chemical compound, small molecule, or other composition, such as an antisense compound, antibody, protease inhibitor, hormone, chemokine or cytokine, capable of inducing a desired therapeutic or prophylactic effect when properly administered to a subject. As used herein, a “candidate agent” is a compound selected for screening to determine if it can function as a therapeutic agent for a particular disease or disorder.

Titer: In the context of the present disclosure, titer refers to the amount of a particular pathogen in a sample.

Toxin: In the context of the present disclosure, “toxin” refers to any poisonous substance, including any chemical toxin or biological toxin.

Transgene: An exogenous nucleic acid sequence introduced into a cell or the genome of an organism.

Transgenic animal: A non-human animal, usually a mammal, having a non-endogenous (heterologous) nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal according to methods well known in the art. A “transgene” is meant to refer to such heterologous nucleic acid, such as, heterologous nucleic acid in the form of an expression construct (such as for the production of a “knock-in” transgenic animal) or a heterologous nucleic acid that upon insertion within or adjacent to a target gene results in a decrease in target gene expression (such as for production of a “knock-out” transgenic animal). A “knock-out” of a gene means an alteration in the sequence of the gene that results in a decrease of function of the target gene, preferably such that target gene expression is undetectable or insignificant. Transgenic knock-out animals can comprise a heterozygous knock-out of a target gene, or a homozygous knock-out of a target gene. “Knock-outs” also include conditional knock-outs, where alteration of the target gene can occur upon, for example, exposure of the animal to a substance that promotes target gene alteration, introduction of an enzyme that promotes recombination at the target gene site (for example, Cre in the Cre-lox system), or other method for directing the target gene alteration postnatally.

Transplant or transplanting: Refers to the process of grafting an organ, tissue or cells from one subject to another subject, or to another region of the same subject.

Urokinase: Also called urokinase-type plasminogen activator (uPA), urokinase is a serine protease. Urokinase was originally isolated from human urine, but is present in several physiological locations, such as the blood stream and the extracellular matrix. The primary physiological substrate is plasminogen, which is an inactive zymogen form of the serine protease plasmin. Activation of plasmin triggers a proteolytic cascade which, depending on the physiological environment, participates in thrombolysis or extracellular matrix degradation. In one embodiment of the methods provided herein, urokinase is administered to a recipient mouse prior to hepatocyte injection. In some embodiments, urokinase is human urokinase. In some embodiments, the human urokinase is the secreted form of urokinase. In some embodiments, the human urokinase is a modified, non-secreted form of urokinase (see U.S. Pat. No. 5,980,886).

Vector: A nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An integrating vector is capable of integrating itself into a host nucleic acid. An expression vector is a vector that contains the necessary regulatory sequences to allow transcription and translation of inserted gene or genes. In one embodiment described herein, the vector comprises a sequence encoding urokinase, such as human urokinase. In one embodiment, the vector is a plasmid vector. In another embodiment, the vector is a viral vector, such as an adenovirus vector or an adeno-associated virus (AAV) vector.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Several Embodiments

Provided herein is a robust method of expanding hepatocytes in vivo. Although human hepatocytes are exemplified herein, hepatocytes from other species can also be expanded, including hepatocytes from rats, dogs, cats, cows, horses, pigs and non-human primates, such as baboons, chimpanzees and rhesus macaques. The method includes transplanting isolated human hepatocytes into an immunodeficient mouse that is deficient for expression of the tyrosine catabolic enzyme fumarylacetoacetate hydrolase (Fah), wherein (i) the mouse is further deficient for expression of IL-1R, or (ii) the mouse is administered an IL-1R antagonist (referred to herein as a “recipient mouse”).

The human hepatocytes are allowed to expand in the recipient mouse for a period of time sufficient to permit expansion of the human hepatocytes. The precise period of time for expansion can be determined empirically with routine experimentation. In some embodiments, the human hepatocytes are allowed to expand for up to 6 months, up to 8 months, up to 10 months or up to 12 months. In other embodiments, the human hepatocytes are allowed to expand for at least about 2 weeks, at least about 4 weeks, at least about 6 weeks, at least about 8 weeks, at least about 12 weeks, at least about 16 weeks, at least about 20 weeks, at least about 24 weeks or at least about 28 weeks. The extent of hepatocyte expansion can vary. In some embodiments, expansion of human hepatocytes in a recipient mouse results in an increase of at least about 10-fold, at least about 50-fold, at least about 100-fold, at least about 150-fold, at least about 200-fold, at least about 250-fold, at least about 300-fold, at least about 400-fold, at least about 500-fold or at least about 1000-fold.

In some embodiments, the immunodeficient and Fah-deficient mouse comprises homozygous disruptions in the Fah gene such that the disruption results in loss of expression of functional FAH protein. The loss of expression of functional FAH protein need not be complete loss of expression. In some examples, loss of expression of functional FAH protein is loss of expression of about 80%, about 90%, about 95% or about 99%. Disruptions in the Fah gene include, for example, an insertion, a deletion or a point mutation in the Fah gene, or any multiple or combination thereof. In particular examples, the disruption comprises a deletion in the Fah gene.

In some embodiments, the immunodeficient and Fah-deficient mouse lacks functional T cells, B cells and NK cells. The immunodeficiency of the mouse can be due to a genetic alteration, immunosuppression, or a combination thereof.

The immunodeficient mouse that is immunodeficient due to a genetic alteration can include any genetic alteration or combination of genetic alterations such that the mouse is impaired in at least one aspect of humoral or cellular immunity. For example, the immunodeficient mouse can have one or more genetic alterations selected from, for example, Rag1 deficiency, Rag2 deficiency, Il2rg deficiency, the SCID mutation, the NOD genotype or the nude mutation.

In some embodiments, the immunodeficient mouse is a Rag2^{-/-}/Il2rg^{-/-} mouse. In other embodiments, the immunodeficient mouse is a Rag1^{-/-}/Il2rg^{-/-} mouse. In some examples the immunodeficient mouse is a NOD/Rag2^{-/-}/Il2rg^{-/-} mouse or a NOD/Rag1^{-/-}/Il2rg^{-/-} mouse. In some embodiments, the Fah-deficient mouse comprises a homozygous deletion of Fah. In other embodiments, the Fah-deficient mouse comprises one or more point mutations in Fah, such that the function and/or production of the protein is substantially reduced. Thus, in some embodiments, the mouse is a Fah^{-/-}/Rag2^{-/-}/Il2rg^{-/-} mouse, a Fah^{-/-}/Rag1^{-/-}/Il2rg^{-/-} mouse, a NOD/Fah^{-/-}/Rag2^{-/-}/Il2rg^{-/-} mouse, or a NOD/Fah^{-/-}/Rag1^{-/-}/Il2rg^{-/-} mouse. In particular examples, the mouse is a Fah^{-/-}/Rag2^{-/-}/Il2rg^{-/-} (FRG) mouse. In other embodiments, the mouse is a Fah^{pm}/Rag2^{-/-}/Il2rg^{-/-} (FP^{pm}RG) mouse.

The immunodeficiency of the mouse can also be due to immunosuppression. Generally, immunosuppression is achieved by administration of one or more immunosuppressants to the mouse, thereby inducing the immunodeficiency. The immunosuppressant or combination of immunosuppressants can be selected from any immunosuppressants known in the art or disclosed herein (for example, see Terms and Methods). Immunosuppressants contemplated for use herein include any compounds that decrease the activity or function of one or more aspects of the immune system, such as the humoral, cellular or complement system.

In some embodiments, the one or more immunosuppressants are selected from FK506, cyclosporin A, fludarabine, mycophenolate, prednisone, rapamycin or azathioprine, or combinations thereof.

The immunosuppressants can be administered to the mouse using any suitable route of delivery, such as by oral administration or intraperitoneal injection. In particular examples, the immunosuppressant is FK506. In some cases, FK506 is administered orally in the drinking water. Suitable doses are known in the art and can be determined by the skilled practitioner. For example, FK506 can be given continuously in the drinking water at a dose of 7.5 mg/L, resulting in an approximate dose of 1 μg per gram per day. However, other suitable doses include about 2.0 to about 15 mg/L, such as about 2.0, about 3.0, about 4.0, about 5.0, about 6.0, about 7.0, about 8.0, about 9.0, about 10.0, about 11.0, about 12.0, about 13.0, about 14.0 or about 15.0 mg/L.

In some examples, the immunosuppressant is cyclosporin A. Cyclosporin A can be, for example, administered in the drinking water, such as at a concentration of about 10 to about 100 mg/kg/day. In some cases, cyclosporine A is administered in the drinking water at a concentration of about 30 to about 70 mg/kg/day, such as about 50 mg/kg/day.

In some examples, the immunosuppressant is fludarabine. Generally, fludarabine is administered by intraperitoneal injection daily. However, administration can occur more or less frequently, such as twice a day, every other day or weekly. Exemplary doses of fludarabine include 100 mg/kg/day to about 500 mg/kg/day. In particular examples, the dose is about 150 to about 250 mg/kg/day, such as about 200 mg/kg/day.

In other embodiments, the mouse is administered a combination of immunosuppressants, such as two or more of FK506, cyclosporin A, fludarabine, azathioprine, mycophenolate and prednisone. In some examples, the combination of immunosuppressants includes FK506, mycophenolate and prednisone. In other examples, the combination of immunosuppressants includes cyclosporin A, mycophenolate and prednisone. In other examples, the combination of immunosuppressants includes rapamycin, mycophenolate and prednisone. In other examples, the combination of immunosuppressants includes azathioprine, FK506 and prednisone. In other examples, the combination of immunosuppressants includes azathioprine, cyclosporin A and prednisone. In other examples, the combination of immunosuppressants includes rapamycin, azathioprine and prednisone.

To improve engraftment efficiency, the immunodeficient and Fah-deficient mouse is either further deficient in IL-1R, or is administered an IL-1R antagonist. In some embodiments in which the mouse is deficient for expression of IL-1R, the mouse is homozygous for disruptions in the Il1r1 gene, such that the disruption results in loss of expression of functional IL-1R protein. The loss of expression of functional IL-1R protein need not be complete loss of expression. In some examples, loss of expression of functional IL-1R protein is loss of expression of about 80%, about 90%, about 95% or about 99%. In some embodiments, the disruption comprises an insertion, a deletion or one or more point mutations in the Il1r1 gene. In some examples, the mouse is a Fah^{-/-}/Rag2^{-/-}/Il2rg^{-/-}/Il1r1^{-/-}, a Fah^{-/-}/Rag1^{-/-}/Il2rg^{-/-}/Il1r1^{-/-} mouse, a NOD/Fah^{-/-}/Rag2^{-/-}/Il2rg^{-/-}/Il1r^{-/-} mouse or a NOD/Fah^{-/-}/Rag1^{-/-}/Il2rg^{-/-}/Il1r1^{-/-} mouse.

In some embodiments, the immunodeficient and Fah-deficient mouse is administered an IL-1R antagonist. The IL-1R antagonist administered to the mouse can be any compound (such as a nucleic acid molecule, polypeptide, antibody or small molecule) that inhibits activity of IL-1R. In some embodiments, the IL-1R antagonist is anakinra In other embodiments, the IL-1R antagonist is IL-1R^{A}, such as an IL-1RA having an amino acid sequences set forth herein as SEQ ID NO: 18 or SEQ ID NO: 20, or a variant or derivative thereof. In some cases, the IL-1R antagonist is delivered at or near the same time hepatocytes are transplanted into the mouse. Additional doses of IL-1R antagonist can be administered following injection of hepatocytes.

IL-1R antagonist can be administered in a single dose or in multiple doses. When multiple doses are used, the dosing schedule can vary. For example, IL-1R antagonist can be administered every hour, twice a day, daily, every other day or weekly. In particular examples, the IL-1R antagonist is administered daily. Daily administration can occur for two days, three days, four days, five days, six days, seven days, eight days, nine days, 10 days or longer. In one example, the IL-1R antagonist is delivered daily for seven days. In another example, the IL-1R antagonist is delivered daily for three days.

The appropriate dose of IL-1R antagonist will vary depending on type of compound that is used and the route of administration. In some embodiments, the IL-1R antagonist is anakinra In particular examples, the total dose of anakinra (whether administered as a single dose, or spread out over several doses) is about 0.2, about 0.4, about 0.6, about 1, about 2, about 3, about 4, about 5, about 6, about 7, about 8, about 9, about 10, about 11, about 12, about 13, about 14, about 15, about 16, about 17, about 18, about 19 or about 20 mg. In some examples, the total dose of anakinra is about 10 to about 20 mg, such as about 12 to about 16 mg. In particular examples, the dose of anakinra is about 14 mg. In other examples, the total dose of anakinra is about 0.2 to about 6 mg, such as about 0.4 to about 2 mg, or about 1 to about 3 mg.

The IL-1R antagonist can be administered to the mouse using any suitable route of administration. In some embodiments, the IL-1R antagonist is administered by injection. For example, the IL-1R antagonist is administered by subcutaneous, intramuscular, intradermal, intraperitoneal or intravenous injection.

In one embodiment of the methods described herein, prior to hepatocyte injection, the Fah-deficient mouse is administered an agent that inhibits, delays or prevents the development of liver disease in the mouse. The agent can be any compound or composition known in the art to inhibit liver disease. On such agent is 2-(2-nitro-4-trifluoro-methyl-benzoyl)-1,3 cyclohexanedione (NTBC). NTBC is administered to regulate the development of liver disease in the Fah-deficient mouse. The dose, dosing schedule and method of administration can be adjusted as needed to prevent liver dysfunction in the Fah-deficient mouse.

In some embodiments, the NTBC is administered at a dose of about 0.01 mg/kg/day to about 2.0 mg/kg/day. In one embodiment, the NTBC is administered at a dose of about 1.0 mg/kg/day to about 2.0 mg/kg/day, such as about 1.4 mg/kg/day to about 1.8 mg/kg/day. In one example, the NTBC is administered at a dose of about 1.6 mg/kg/day. In one embodiment, the NTBC is administered at a dose of about 0.01 mg/kg/day to about 0.50 mg/kg/day. In another embodiment, the NTBC is administered at a dose of about 0.05 mg/kg/day to about 0.10 mg/kg/day, such as about 0.05 mg/kg/day, about 0.06 mg/kg/day, about 0.07 mg/kg/day, about 0.08 mg/kg/day, about 0.09 mg/kg/day or about 0.10 mg/kg/day. NTBC can be administered prior to injection of human hepatocytes and/or a selected period of time following hepatocyte injection. NTBC can be withdrawn or re-administered as needed during the time of hepatocyte expansion. In one embodiment, the Fah-deficient mouse is administered NTBC prior to hepatocyte injection and for at least about three days following hepatocyte injection. In another embodiment, the Fah-deficient mouse is administered NTBC prior to hepatocyte injection and for at least about six days following hepatocyte injection. In one aspect, the dose of NTBC is gradually reduced over the course of a six day period following hepatocyte injection.

NTBC can be administered by any suitable means, such as, but limited to, in the drinking water, in the food or by injection. In one embodiment, the concentration of NTBC administered in the drinking water prior to hepatocyte injection is about 1 to about 8 mg/L, such as about 1 mg/L, about 2 mg/L, about 3 mg/L, about 4 mg/L, about 5 mg/L, about 6 mg/L, about 7 mg/L or about 8 mg/L. In another embodiment, the concentration of NTBC administered in the drinking water prior to hepatocyte injection is about 1 to about 2 mg/L, such as about 1.0 mg/L, about 1.2 mg/L, about 1.4 mg/L, about 1.6 mg/L, about 1.8 mg/L or about 2.0 mg/L.

Disclosed herein is a method of expanding human hepatocytes in vivo comprising transplanting human hepatocytes, such as by injection, into an immunodeficient and Fah-deficient mouse (also referred to as a recipient mouse), administering an IL-1R antagonist to the mouse and allowing the human hepatocytes to expand. In some embodiments, the method further includes collecting the expanded human hepatocytes from the mouse. Alternatively, the method of expanding human hepatocytes includes transplanting human hepatocytes into an immunodeficient, Fah-deficient and IL-1R-deficient mouse (this type of mouse is also a “recipient mouse”) and allowing the human hepatocytes to expand. In some embodiments, this method further includes collecting the expanded human hepatocytes from the mouse.

In some embodiments, the human hepatocytes transplanted into the recipient mouse are isolated human hepatocytes. In other embodiments, the human hepatocytes are transplanted as part of a liver tissue.

The hepatocytes can be transplanted using any suitable means known in the art. In one embodiment, the human hepatocytes are transplanted, such as by injection, into the spleen of the recipient mouse. In another embodiment, the expanded human hepatocytes are collected from the liver of the recipient mouse.

Also provided is a method of expanding human hepatocytes in vivo wherein a recipient mouse is administered a vector encoding a urokinase gene prior to injection of the human hepatocytes. In one embodiment, the urokinase gene is human urokinase. Wild-type urokinase is a secreted protein. Thus, in some embodiments, the human urokinase is a secreted form of urokinase (Nagai et al., Gene 36:183-188, 1985). Sequences for human urokinase (secreted form) are known in the art, such as, but not limited to the GenBank Accession Nos. AH007073 (deposited Aug. 3, 1993), D11143 (deposited May 9, 1996), A18397 (deposited Jul. 21, 1994), BC002788 (deposited Aug. 19, 2003), X02760 (deposited Apr. 21, 1993), BT007391 (deposited May 13, 2003), NM_{—}002658 (deposited Oct. 1, 2004) and X74039 (deposited Feb. 20, 1994).

In some embodiments, the human urokinase is a modified, non-secreted form of urokinase. For example, Lieber et al. (Proc. Natl. Acad. Sci. 92:6210-6214, 1995) describe non-secreted forms of urokinase generated by inserting a sequence encoding an endoplasmic reticulum retention signal at the carboxyl terminus of urokinase, or by replacing the pre-uPA signal peptide with the amino-terminal RR-retention signal (Strubin et al., Cell 47:619-625, 1986; Schutze et al., EMBO J. 13:1696-1705, 1994) and the transmembrane anchor separated by a spacer peptide from the membrane II protein Iip33 (Strubin et al., Cell 47:619-625, 1986). Non-secreted forms of urokinase are also described in U.S. Pat. No. 5,980,886.

The vector encoding urokinase can be any type of vector suitable for delivery to a mouse and capable of expressing the urokinase gene. Such vectors include viral vectors or plasmid vectors. In one embodiment, the vector is an adenovirus vector. In another embodiment, the vector is an AAV vector. The vector encoding urokinase can be administered by any suitable means known in the art. In one embodiment, the vector is administered intravenously. In one aspect, the vector is administered by retroorbital injection. The vector encoding urokinase can be administered any time prior to injection of the human hepatocytes. Typically, the vector is administered to allow sufficient time for urokinase to be expressed. In one embodiment, the vector is administered 24 to 48 hours prior to hepatocyte injection.

Further provided herein is a method of expanding human hepatocytes in vivo wherein the recipient mouse is depleted of macrophages prior to injection of the human hepatocytes. In one embodiment, the recipient mouse is administered a vector encoding urokinase prior to macrophage depletion. In another embodiment, the recipient mouse is administered a vector encoding urokinase following macrophage depletion. In another embodiment, the macrophage-depleted recipient mouse is not administered a vector encoding urokinase. Macrophages can be depleted from the recipient mouse using any one of a number of methods well known in the art, such as by using a chemical or an antibody. For example, macrophages can be deleted by administration of an antagonist, such as a toxic substance, including C12MDP, or antibodies altering macrophage development, function and/or viability. The administration of antagonists is performed by well-known techniques, including the use of liposomes, such as described in European Patent No. 1552740. Clodronate-containing liposomes also can be used to deplete macrophages as described by van Rijn et al. (Blood 102:2522-2531, 2003).

In some embodiments, the human hepatocytes transplanted into the immunodeficient and Fah-deficient mouse are isolated human hepatocytes. In some embodiments, the human hepatocytes are transplanted as part of a liver tissue graft. The isolated human hepatocytes can be obtained from any one of a number of different sources. In one embodiment, the human hepatocytes were isolated from the liver of an organ donor. In another embodiment, the human hepatocytes were isolated from a surgical resection. In another embodiment, the human hepatocytes were derived from a stem cell, such as an embryonic stem cell, an iPS cell, a mesenchymal-derived stem cell, an adipose tissue-derived stem cell, a multipotent adult progenitor cells, an unrestricted somatic stem cell, or tissue-specific liver stem cells, which can be found in the liver itself, the gall bladder, intestine or pancreas. In another embodiment, the human hepatocytes were derived from monocytes or amniocytes, thus a stem cell or progenitor cell is obtained in vitro to produce hepatocytes. In another embodiment, the human hepatocytes were cryopreserved prior to injection.

Further provided herein is a method of serial transplantation of human hepatocytes in the Fah-deficient recipient mouse. The method comprises collecting the expanded human hepatocytes from a first recipient mouse and further expanding the hepatocytes in a second, third, fourth or additional recipient mouse (Overturf et al., Am. J. Pathol. 151: 1078-9107, 1997). Human hepatocytes can be collected from a mouse using any one of a number of techniques. For example, the hepatocytes can be collected by perfusing the mouse liver, followed by gentle mincing, as described in the Examples below. Furthermore, the hepatocytes can be separated from other cell types, tissue and/or debris using well known methods, such as by using an antibody that specifically recognizes human cells, or human hepatocytes. Such antibodies include, but are not limited to an antibody that specifically binds to a class I major histocompatibility antigen, such as anti-human HLA-A,B,C (Markus et al. Cell Transplantation 6:455-462, 1997). Antibody bound hepatocytes can then be separated by panning (which utilizes a monoclonal antibody attached to a solid matrix), fluorescence activated cell sorting (FACS), magnetic bead separation or the like. Alternative methods of collecting hepatocytes are well known in the art.

In some embodiments, the methods provided herein further include collecting a biological sample from the mouse. For example, a biological sample can be a biological fluid, cell or tissue sample. In some examples, the biological sample is a fluid sample, such as a blood or urine sample. In some cases, the method includes collecting the expanded human hepatocytes from the recipient mouse and further collecting a biological sample from the mouse, such as a blood or urine sample.

A method for selecting an agent effective for the treatment of a human liver disease is also provided. In some embodiments, the method includes (i) administering a candidate agent to an immunodeficient and Fah-deficient mouse transplanted with human hepatocytes, wherein the mouse is further deficient for expression of IL-1R, or the mouse is administered an IL-1R antagonist; and (ii) assessing the effect of the candidate agent on the liver disease. An improvement in one or more signs or symptoms of the liver disease, indicates the candidate agent is effective for the treatment of the liver disease. In some embodiments, the liver disease is hepatic infection, fibrosis, cirrhosis or liver cancer, such as HCC.

Also provided is a method for selecting an agent effective for the treatment of infection by a human hepatic pathogen. In some embodiments, the method includes (i) administering a candidate agent to the immunodeficient and Fah-deficient mouse transplanted with human hepatocytes, wherein the mouse is further deficient for expression of IL-1R, or the mouse is administered an IL-1R antagonist, and wherein the transplanted human hepatocytes of the immunodeficient and Fah-deficient mouse are infected with the hepatic pathogen; and (ii) assessing the effect of the candidate agent on the hepatic infection. A decrease in infectious load of the hepatic pathogen relative to infectious load in the Fah-deficient mouse prior to administration of the candidate agent, indicates the candidate agent is effective for the treatment of infection by the hepatic pathogen.

In some embodiments, the infectious load is determined by measuring titer of the pathogen in a sample obtained from the mouse. In some embodiments, the infectious load is determined by measuring a pathogen-specific antigen in a sample obtained from the mouse. In some embodiments, the infectious load is determined by measuring a pathogen-specific nucleic acid molecule in a sample obtained from the mouse. In some embodiments, the hepatic pathogen is a hepatotropic virus, such as HBV or HCV.

Further provided is a method for selecting an agent effective for the treatment of cirrhosis. In some embodiments, the method includes (i) administering a candidate agent to the immunodeficient and Fah-deficient mouse transplanted with human hepatocytes, wherein the mouse is further deficient for expression of IL-1R, or the mouse is administered an IL-1R antagonist, and wherein the immunodeficient and Fah-deficient mouse has been administered a compound that induces the development of cirrhosis in the mouse; and (ii) assessing the effect of the candidate agent on at least one diagnostic marker of cirrhosis in the immunodeficient and Fah-deficient mouse, wherein the at least one diagnostic marker of cirrhosis is selected from AST, ALT, bilirubin, alkaline phosphatase and albumin. A decrease in AST, ALT, bilirubin or alkaline phosphatase, or an increase in albumin in the Fah-deficient mouse relative to the Fah-deficient mouse prior to administration of the candidate agent, indicates the candidate agent is effective for the treatment of cirrhosis.

Also provided is a method for selecting an agent effective for the treatment of hepatocellular carcinoma (HCC). In some embodiments, the method includes (i) administering a candidate agent to the immunodeficient and Fah-deficient mouse transplanted with human hepatocytes, wherein the mouse is further deficient for expression of IL-1R, or the mouse is administered an IL-1R antagonist, and wherein the immunodeficient and Fah-deficient mouse has been administered a compound that induces the development of HCC in the mouse or has been transplanted with malignant hepatocytes; and (ii) assessing the effect of the candidate agent on HCC in the immunodeficient and Fah-deficient mouse. A decrease in tumor growth or tumor volume in the mouse relative to the mouse prior to administration of the candidate agent, indicates the candidate agent is effective for the treatment of HCC.

Further provided is a method of assessing the effect of an exogenous agent on human hepatocytes in vivo. In some embodiments, the method includes (i) administering the exogenous agent to an immunodeficient and Fah-deficient mouse transplanted with human hepatocytes, wherein the mouse is further deficient for expression of IL-1R, or the mouse is administered an IL-1R antagonist; and (ii) measuring at least one marker of liver function in the immunodeficient and Fah-deficient mouse, wherein the at least one marker of liver function is selected from AST, ALT, bilirubin, alkaline phosphatase and albumin. An increase in AST, ALT, bilirubin or alkaline phosphatase, or a decrease in albumin in the mouse, relative to the mouse prior to administration of the exogenous agent, indicates the exogenous agent is toxic. In some embodiments, the exogenous agent is a known or suspected toxin.

### IV. Interleukin-1 Receptor (IL-1R) Antagonists

It is disclosed herein that administration of an IL-1R antagonist significantly enhances engraftment of human hepatocytes in immunodeficient mice having a functional deletion in Fah. Thus, provided herein are methods for engrafting and expanding human hepatocytes in immunodeficient/Fah-deficient mice, wherein in some embodiments, the methods include administering to the mice an IL-1R antagonist. Any compound (such as a nucleic acid molecule, polypeptide, antibody or small molecule) that functions as an IL-1R antagonist is contemplated for use herein. Also contemplated are the use of immunodeficient and Fah-deficient mice that are further deficient in IL-1R.

As used herein, the term “IL-1 receptor antagonist” refers to any molecule that binds to IL-1R or IL-1R accessory protein (IL-1RAP) and interferes with the activation mediated by IL-1R, for example by inhibiting or preventing the interaction of IL-1R and IL-1RAP, or the interaction of IL-1 and IL-1R. In some embodiments, the IL-1R antagonist is anakinra In other embodiments, the IL-1R antagonist is IL-1RA (such as human or mouse IL-1RA set forth herein as SEQ ID NOs: 18 and 20, respectively), or a functional variant thereof, such as a conservative variant of IL-1RA. In other embodiments, the IL-1R antagonist is an antibody specific for IL-1R (such as an antibody specific for IL-1R type I), an antibody specific for IL-1RAP, a peptide that binds to IL-1R type I, or a peptide that binds to IL-1RAP.

IL-1R antagonists, including IL-1RA and variants and derivatives thereof, have been previously described (see, for example, PCT Publication Nos. WO 91/08285; WO 91/17184; WO 92/16221; WO 93/21946; WO 94/06457; WO 94/21275; WO 94/21235; WO 94/20517; WO 96/22793; WO 97/28828; and WO 99/36541; U.S. Pat. Nos. 5,075,222 and 6,599,873; 6,268,142; 6,168,791; 6,159,460; 6,090,775; 6,063,600; 6,036,978; 6,054,559; 5,922,573; 5,863,769; 5,858,355; 5,863,769; 5,508,262; 6,013,253; and 6,399,573; and U.S. Patent Application Publication Nos. 2004/0076991 and 2005/0282752).

For purposes of the present disclosure, the term “IL-1RA” includes modified forms of IL-1RA in which amino acids of IL-1RA have been deleted, inserted or substituted. It will be appreciated by those skilled in the art that many combinations of deletions, insertions and substitutions can be made within the amino acid sequences of IL-1RA (such as the amino acid sequences set forth herein as SEQ ID NO: 18 and SEQ ID NO: 20), provided that the resulting molecule is biologically active (e.g., possesses the ability to inhibit IL-1R).

The term “IL-1 receptor antagonist” also includes fusion proteins comprising IL-1RA. Exemplary fusion proteins include Fc-IL-1RA and other fusion molecules described in the art (see, for example, U.S. Patent Application Publication No. 2007/0248597 and U.S. Pat. No. 6,294,170).

In some embodiments, the IL-1R antagonist is an antibody specific for IL-1R type I (IL-1R1). Examples of IL-1R1 antibodies are described in the art (see, for example, U.S. Patent Application Publication No. 2004/0097712) and are commercially available from a variety of sources. Additional IL-1R1 antibodies can be produced according to well known methods. As used herein, “antibody” refers to a complete immunoglobulin molecule or an antibody fragment, such as Fab fragments, Fab′ fragments, F(ab)′₂ fragments, single chain Fv proteins (scFv), and disulfide stabilized Fv proteins (dsFv). Antibodies can be, for example, chimeric antibodies, murine antibodies or humanized antibodies. Both polyclonal and monoclonal antibodies are contemplated for use with the disclosed methods.

In some embodiments, a nucleic acid molecule encoding an IL-1R antagonist is delivered to a recipient mouse. In some embodiments, the nucleic acid molecule encodes anakinra In other embodiments, the nucleic acid molecule encodes IL-1RA, or a variant or derivative of IL-1RA. In particular examples, the nucleic acid molecule comprises SEQ ID NO: 17 or SEQ ID NO: 19. In some cases, the nucleic acid molecule comprises a vector, such as a plasmid or viral vector. Suitable plasmid and viral vectors for delivery of heterologous nucleic acid sequences are well known in the art and are described herein below (such as those vectors described below for delivery of urokinase).

### V. Genetically Modified Mice for Expansion of Human Hepatocytes

Several groups have attempted to engraft and expand primary human hepatocytes in rodents (U.S. Pat. No. 6,509,514; PCT Publication No. WO 01/07338; U.S. Publication No. 2005-0255591). Dandri et al. (Hepatology 33:981-988, 2001) were the first to report successful repopulation of mouse livers with human hepatocytes. Since then, other groups have reported successful engraftment of human liver cells in mice. In all of these studies, the animals used were transgenic animals expressing urokinase plasminogen activator (uPA) under the transcriptional control of an albumin promoter (Sandgren et al. Cell 66:245-256, 1991). Overexpression of uPA causes metabolic disruption, leading to cell death of the mouse hepatocytes without affecting the transplanted human hepatocytes, which do not express the transgene. The alb-uPA transgene was crossed onto various immune deficient backgrounds to prevent rejection of the human cells (Tateno et al. Am. J. Pathol. 165:901-912, 2004; Katoh et al. J. Pharm. Sci. 96:428-437, 2007; Turrini et al. Transplant. Proc. 38:1181-1184, 2006).

While engraftment levels of up to 90% have been reported in these models, the system has several major disadvantages which have prevented wide-spread use. First, the alb-uPA transgene becomes inactivated or lost early in life. For this reason, it is necessary to transplant human cells very early (14 days of age) and to use mice which are homozygous for the transgene. This narrow transplantation time window severely restricts the flexibility of the model. Second, the spontaneous inactivation of the transgene creates a pool of transgene-negative, healthy mouse hepatocytes. These revertant murine hepatocytes compete efficiently with human cells during repopulation. It is therefore not possible to repopulate secondary recipients upon serial transplantation of the human cells. Third, liver disease has a very early onset in this model, thus reducing the viability of the transgenic mice. Consequently, it is difficult to breed sufficient numbers of experimental animals. In addition, the transgenic mice have a bleeding tendency which increases mortality during surgery. Finally, alb-uPA transgenic animals develop renal disease once the repopulation with human cells exceeds 50%. This is thought to be due to the action of human complement on renal epithelium. To obtain very high levels of human engraftment it is necessary to treat the transplanted mice with an anti-complement protease inhibitor (Tateno et al. Am. J. Pathol. 165:901-912, 2004). Because of these many limitations, a more robust system for expanding human hepatocytes is highly desirable.

Described herein is a highly efficient method for expanding human hepatocytes in vivo using a genetically modified mouse having a unique combination of gene deletions. Successful engraftment and expansion of human hepatocytes in mouse liver requires an immunodeficient mouse with some degree of liver dysfunction. Mouse livers have been repopulated with human hepatocytes in a variety of different types of immunodeficient mice, including RAG-2 knockout or SCID mice, both of which lack B cells and T cells (U.S. Pat. No. 6,509,514; PCT Publication No. WO 01/07338; U.S. Publication No. 2005-0255591). To achieve liver dysfunction, immunodeficient mice were crossed with urokinase plasminogen activator (uPA) transgenic mice. Expression of uPA in the mouse liver creates a growth disadvantage for the mouse hepatocytes, which facilitates the expansion of transplanted human hepatocytes (PCT Publication No. WO 01/07338). To avoid the limitations of the uPA transgene, Fah-deficient mice were analyzed for their capacity to allow for expansion of human hepatocytes. FAH is a metabolic enzyme that catalyzes the last step of tyrosine catabolism. Mice having a homozygous deletion of the Fah gene exhibit altered liver mRNA expression and severe liver dysfunction (Grompe et al. Genes Dev. 7:2298-2307, 1993).

It is disclosed herein that Fah^{-/-}/Rag2^{-/-}/Il2rg^{-/-} (FRG) triple mutant mice lack T cells, B cells and NK cells. Rag2^{-/-}/Il2rg^{-/-} mice are known in the art (Traggiai et al. Science 304:104-107, 2004; Gorantla et al. J. Virol. 81:2700-2712, 2007).

As described in the Examples below, engraftment and expansion of human hepatocytes is surprisingly highly efficient in FRG mice. For example, an FRG mouse can be injected with one million isolated human hepatocytes. Assuming 10% efficiency, 100,000 human hepatocytes engraft in the recipient mouse. An average yield from an FRG mouse following expansion is then about 80 to about 120 million human hepatocytes, which equates to an 800- to 1.200-fold increase in human hepatocytes. FRG mice can also be used for serial transplantation of human hepatocytes. Serial transplantation can involve multiple mice and can result in at least about 1.000-fold expansion of human hepatocytes per mouse.

Any immunodeficient mouse comprising Fah-deficiency is suitable for the methods described herein. In one embodiment, the mouse is a Rag2^{-/-}/Il2rg^{-/-} mouse which is also deficient in Fah. In another embodiment, the mouse is a Rag1^{-/-}/Il2rg^{-/-} mouse. In other embodiments, the mouse is a NOD/Rag^{-/-}/Il2rg^{-/-} mouse or a NOD/Rag1^{-/-}/Il2rg^{-/-} mouse. Although some specific combinations of genetic alterations are described herein, other combinations of genetic alterations resulting in immunodeficiency are contemplated herein. For example, other genetic alterations include the SCID mutation, the nude mutation and NOD genotype.

The Fah-deficient mouse can comprise, for example, homozygous deletions in Fah, or one or more point mutations in Fah. Fah-deficiency (such as by point mutation or homozygous deletion) results in a substantial decrease in, or the absence of, Fah mRNA expression and/or functional FAH protein. In addition to the FRG mouse, it is described herein that an immunodeficient mouse (Rag2^{-/-}/Il2rg^{-/-}) homozygous for a point mutation in the Fah gene (referred to herein as the F^{pm}RG mouse) also is a suitable mouse for engraftment and expansion of human hepatocytes in vivo. Also contemplated herein is the use of an immunodeficient, Fah-deficient and IL-1R-deficient mouse. IL-1R-deficient mice have been previously described (see, for example, Norman et al., Ann. Surg. 223(2):163-169, 1996; Glaccum et al., J. Immunol. 159:3364-3371, 1997) and are commercially available (e.g., strains B6;129S1-Il1r1^{tm1Rom1}/J and B6.129S7-Il1r1^{tm1Imx}/J) from The Jackson Laboratory (Bar Harbor, Me.).

### VI. Isolation and Delivery of Human Hepatocytes

A significant advantage of using Fah-deficient mice for the in vivo expansion of human hepatocytes is the ability to engraft the mice with human hepatocytes derived from a variety of sources. Any suitable source of human hepatocytes or hepatocyte precursors/progenitors can be used in the disclosed methods for transplantation in Fah-deficient mice. As described in the Examples below, human hepatocytes can be derived from cadaveric donors or liver resections, or can be obtained from commercial sources. In addition, as shown herein, FRG mice can be successfully transplanted with human hepatocytes from donors of all ages or with cryopreserved hepatocytes. There is often a delay (typically 1 to 2 days) between isolation of human hepatocytes and transplantation, which can result in poor viability of the hepatocytes. However, the FRG mouse system is capable of expanding human hepatocytes even when engrafted with hepatocytes of limited viability.

Methods of isolating human hepatocytes are well known in the art. For example, methods of isolating human hepatocytes from organ donors or liver resections are described in PCT Publication Nos. WO 2004/009766 and WO 2005/028640 and U.S. Pat. Nos. 6,995,299 and 6,509,514. Hepatocytes can be obtained from a liver biopsy taken percutaneously or via abdominal surgery. Human hepatocytes for transplantation into a recipient animal, such as an FRG mouse, are isolated from human liver tissue by any convenient method known in the art. Liver tissue can be dissociated mechanically or enzymatically to provide a suspension of single cells, or fragments of intact human hepatic tissue may be used. For example, the hepatocytes are isolated from donor tissue by routine collagenase perfusion (Ryan et al. Meth. Cell Biol. 13:29, 1976) followed by low-speed centrifugation. Hepatocytes can then be purified by filtering through a stainless steel mesh, followed by density-gradient centrifugation. Alternatively, other methods for enriching for hepatocytes can be used, such as, for example, fluorescence activated cell sorting, panning, magnetic bead separation, elutriation within a centrifugal field, or any other method well known in the art. Similar hepatocyte isolation methods can be used to collect expanded human hepatocytes from recipient mouse liver.

Alternatively, human hepatocytes can be prepared using the technique described by Guguen-Guillouzo et al. (Cell Biol. Int. Rep. 6:625-628, 1982). Briefly, a liver or portion thereof is isolated and a cannula is introduced into the portal vein or a portal branch. The liver tissue is then perfused, via the cannula, with a calcium-free buffer followed by an enzymatic solution containing collagenase (such as about 0.025% collagenase) in calcium chloride solution (such as about 0.075% calcium chloride) in HEPES buffer at a flow rate of between 30 and 70 milliliters per minute at 37° C. The perfused liver tissue is minced into small (such as about 1 cubic millimeter) pieces. The enzymatic digestion is continued in the same buffer as described above for about 10-20 minutes with gentle stirring at 37° C. to produce a cell suspension. The released hepatocytes are collected by filtering the cell suspension through a 60-80 micrometer nylon mesh. The collected hepatocytes can then be washed in cold HEPES buffer at pH 7.0 using slow centrifugation to remove collagenase and cell debris. Non-parenchymal cells may be removed by metrizamide gradient centrifugation (see U.S. Pat. No. 6,995,299).

Human hepatocytes can be obtained from fresh tissue (such as tissue obtained within hours of death) or freshly frozen tissue (such as fresh tissue frozen and maintained at or below about 0° C.). Preferably, the human tissue has no detectable pathogens, is normal in morphology and histology, and is essentially disease-free. The hepatocytes used for engraftment can be recently isolated, such as within a few hours, or can be transplanted after longer periods of time if the cells are maintained in appropriate storage media. One such media described in the Examples below is VIASPAN™ (a universal aortic flush and cold storage solution for the preservation of intra-abdominal organs; also referred to as University of Wisconsin solution, or UW). Hepatocytes also can be cryopreserved prior to transplantation. Methods of cryopreserving hepatocytes are well known in the art and are described in U.S. Pat. No. 6,136,525.

In addition to obtaining human hepatocytes from organ donors or liver resections, the cells used for engraftment can be human stem cells or hepatocyte precursor cells which, following transplantation into the recipient animal, develop or differentiate into human hepatocytes capable of expansion. Human cells with ES cell properties have been isolated from the inner blastocyst cell mass (Thomson et al., Science 282:1145-1147, 1998) and developing germ cells (Shamblott et al., Proc. Natl. Acad. Sci. USA 95:13726-13731, 1998), and human embryonic stem cells have been produced (see U.S. Pat. No. 6,200,806). As disclosed in U.S. Pat. No. 6,200,806, ES cells can be produced from human and non-human primates. iPS induced from human and non-human primate cells can also be obtained (see, for example, Yu et al., Science 318(5858):1917-1920, 2007; Takahashi et al., Cell 131(5):861-872, 2007; Liu et al., Cell Stem Cell 3(6):587-590, 2008). Generally, primate ES cells are isolated on a confluent layer of murine embryonic fibroblast in the presence of ES cell medium. ES medium generally consists of 80% Dulbecco's modified Eagle's medium (DMEM; no pyruvate, high glucose formulation, Gibco BRL), with 20% fetal bovine serum (FBS; Hyclone), 0.1 mM β-mercaptoethanol (Sigma), 1% non-essential amino acid stock (Gibco BRL). Distinguishing features of ES cells, as compared to the committed “multipotential” stem cells present in adults, include the capacity of ES cells to maintain an undifferentiated state indefinitely in culture, and the potential that ES cells have to develop into every different cell types. Human ES (hES) express SSEA-4, a glycolipid cell surface antigen recognized by a specific monoclonal antibody (see, for example, Amit et al., Devel. Biol. 227:271-278, 2000).

Human hepatocytes derived from human mesenchymal stem cells (hMSCs) can also be used in the methods described herein. Sequential exposure of bone marrow-derived hMSCs to hepatogenic factors results in differentiation of the stem cells to cells with hepatocyte properties (see Snykers et al. BMC Dev Biol. 7:24, 2007; Aurich et al. Gut. 56(3):405-15, 2007). Hepatogenic differentiation of bone marrow-derived mesenchymal stem cells and adipose tissue-derived stem cells (ADSCs) has also been described (see Talens-Visconti et al. World J Gastroenterol. 12(36):5834-45, 2006). Human hepatocytes can also be generated from monocytes. Ruhnke et al. (Transplantation 79(9):1097-103, 2005) describe the generation of hepatocyte-like (NeoHep) cells from terminally differentiated peripheral blood monocytes. The NeoHep cells resemble primary human hepatocytes with respect to morphology, expression of hepatocyte markers, various secretory and metabolic functions and drug detoxification activities. In addition, human hepatocytes derived from amniocytes, also can be used in the methods described herein.

Human ES cell lines exist and can be used in the methods disclosed herein. Human ES cells can also be derived from preimplantation embryos from in vitro fertilized (IVF) embryos. Experiments on unused human IVF-produced embryos are allowed in many countries, such as Singapore and the United Kingdom, if the embryos are less than 14 days old. Only high quality embryos are suitable for ES isolation. Present defined culture conditions for culturing the one cell human embryo to the expanded blastocyst have been described (see Bongso et al., Hum Reprod. 4:706-713, 1989). Co-culturing of human embryos with human oviductal cells results in the production of high blastocyst quality. IVF-derived expanded human blastocysts grown in cellular co-culture, or in improved defined medium, allows isolation of human ES cells (see U.S. Pat. No. 6,200,806).

In one embodiment, human hepatocytes are delivered to recipient mice by transplantation, such as by injection, into the spleen. Hepatocytes can be delivered by other means, such as by injection into liver parenchyma or the portal vein. The number of human hepatocytes injected into a recipient mouse can vary. In one embodiment, about 10⁵ to about 10⁷ human hepatocytes are injected. In another embodiment, about 5×10⁵ to about 5×10⁶ human hepatocytes are injected. In one exemplary embodiment, about 10⁶ human hepatocytes are injected.

### VII. Fah-deficient Mice and Uses Thereof

The Fah-deficient mice disclosed herein can be used for a variety of research and therapeutic purposes. For example, hepatocytes (such as human hepatocytes) expanded in immunodeficient and Fah-deficient mice can be used for studies of drug metabolism and toxicity, as well as hepatic pathogen infection. Although human hepatocytes are exemplified herein, hepatocytes from other species, including rats, dogs, cats, cows, pigs, horses or non-human primates, such as baboons, chimpanzees and rhesus macaques, can also be expanded in Fah-deficient mice. Human hepatocytes expanded in Fah-deficient mice can be used to reconstitute human liver in a subject in need of such therapy. In addition, Fah-deficient mice reconstituted with human hepatocytes can serve as animal models of liver disease, such as HCC, cirrhosis and hepatic infection. Exemplary uses of Fah-deficient mice, and hepatocytes expanded in Fah-deficient mice, are discussed further below.

A. Expansion of Human Hepatocytes and their Medical Use

The present disclosure contemplates the use of human hepatocytes expanded in and collected from recipient mice as a source of human hepatocytes for liver reconstitution in a subject in need of such therapy. Reconstitution of liver tissue in a patient by the introduction of hepatocytes is a potential therapeutic option for patients with acute liver failure, either as a temporary treatment in anticipation of liver transplant or as a definitive treatment for patients with isolated metabolic deficiencies (Bumgardner et al. Transplantation 65: 53-61, 1998). Hepatocyte reconstitution may be used, for example, to introduce genetically modified hepatocytes for gene therapy or to replace hepatocytes lost as a result of disease, physical or chemical injury, or malignancy (U.S. Pat. No. 6,995,299). In addition, expanded human hepatocytes can be used to populate artificial liver assist devices. Methods of collecting human hepatocytes from Fah-deficient mice, as well medical uses of the expanded human hepatocytes, are described in greater detail below.

1. Collecting Human Hepatocytes from Fah-Deficient Mice

Human hepatocytes can be collected from recipient mice using any of a number of techniques known in the art. For example, mice can be anesthetized and the portal vein or inferior vena cava cannulated with a catheter. The liver can then be perfused with an appropriate buffer (such as a calcium- and magnesium-free EBSS supplemented with 0.5 mM EGTA and 10 mM HEPES), followed by collagenase treatment (for example, using a solution was of EBSS supplemented with 0.1 mg/ml collagenase XI and 0.05 mg/ml DNase I). The liver can be gently minced and filtered through nylon mesh (such as sequentially through 70 μm and 40 μm nylon mesh), followed by centrifugation and washing of the cells.

Human hepatocytes collected from recipient mice can be separated from non-human cells or other contaminants (such as tissue or cellular debris) using any technique well known in the art. For example, such methods include using an antibody which selectively binds to human hepatocytes. Such antibodies include, but are not limited to an antibody that specifically binds to a class I major histocompatibility antigen, such as anti-human HLA-A,B,C (Markus et al. Cell Transplantation 6:455-462, 1997) or CD46. Antibodies specific for human cells or human hepatocytes can be used in a variety of different techniques, including FACS, panning or magnetic bead separation. Alternatively, antibodies which bind selectively to mouse cells can be used to remove contaminating mouse cells and thereby enrich human hepatocytes. FACS employs a plurality of color channels, low angle and obtuse light-scattering detection channels, and impedance channels, among other more sophisticated levels of detection, to separate or sort cells (see U.S. Pat. No. 5,061,620) bound by the antibody. Magnetic separation involves the use of paramagnetic particles which are: 1) conjugated to the human specific antibodies; 2) conjugated to detection antibodies which are able to bind to the human specific antibodies; or 3) conjugated to avidin which can bind to biotinylated antibodies. Panning involves a monoclonal antibody attached to a solid matrix, such as agarose beads, polystyrene beads, hollow fiber membranes or plastic petri dishes. Cells that are bound by the antibody can be isolated from a sample by simply physically separating the solid support from the sample.

Hepatocytes collected from Fah-deficient mice can be, for example, cryopreserved for later use, or plated in tissue culture for shipping and future use.

2. Human Liver Reconstitution

Fah-deficient mice provide a system for propagating human hepatocytes that can be used to reconstitute a human liver, as an alternative or adjunct to liver transplant. Currently, patients suffering from liver disease may have to wait for long periods of time before a suitable organ for transplant becomes available. After transplant, patients need to be treated with immunosuppressive agents for the duration of their lives in order to avoid rejection of the donor's liver. A method for propagating the patient's own cells could provide a source of functional liver tissue which would not require immunosuppression to remain viable. Accordingly, the immunodeficient and Fah-deficient mice disclosed herein can be used to reconstitute the liver of a subject with liver disease and/or liver failure using their own hepatocytes, including those produced from patient specific stem cells, that have been expanded in the Fah-deficient mice, or hepatocytes from a donor.

Reconstitution of liver tissue in a patient by the introduction of hepatocytes (also referred to as “hepatocyte transplantation”) is a potential therapeutic option for patients with acute liver failure, either as a temporary treatment in anticipation of liver transplant or as a definitive treatment for patients with isolated metabolic deficiencies (Bumgardner et al., Transplantation 65: 53-61, 1998). A major obstacle to achieving therapeutic liver reconstitution is immune rejection of transplanted hepatocytes by the host, a phenomenon referred to (where the host and donor cells are genetically and phenotypically different) as “allograft rejection.” Immunosuppressive agents have been only partially successful in preventing allograft rejection (Javregui et al., Cell Transplantation 5: 353-367, 1996; Makowka et al., Transplantation 42: 537-541, 1986). Human hepatocytes expanded in Fah-deficient mice may also be used for gene therapy applications. In the broadest sense, such hepatocytes are transplanted into a human host to correct a genetic defect. The passaged hepatocytes need not, but can be derived originally from the same individual who is to be the recipient of the transplant.

In some embodiments, human hepatocytes expanded in Fah-deficient mice may be used to reconstitute liver tissue in a subject as a prelude or an alternative to liver transplant. As one non-limiting example, a subject suffering from progressive degeneration of the liver, for example, as a result of alcoholism, may serve as a donor of hepatocytes which are then expanded in a Fah-deficient mouse. The number of hepatocytes is expanded relative to the number originally obtained from the subject and transplanted into the Fah-deficient mouse. Following expansion, the human hepatocytes can be isolated from the Fah-deficient mouse and can be used to reconstitute the subject's liver function. Expanding hepatocytes in Fah-deficient mice may be used not only to increase the number of hepatocytes, but also to selectively remove hepatocytes that are afflicted with infectious or malignant disease. Specifically, a subject may be suffering from viral hepatitis, where some but not all of the hepatocytes are infected and infected hepatocytes can be identified by the presence of viral antigens in or on the cell surface. In such an instance, hepatocytes can be collected from the subject, and non-infected cells can be selected for expanding in one or more Fah-deficient mice, for example by FACS. Meanwhile, aggressive steps could be taken to eliminate infection in the patient. Following treatment, the subject's liver tissue may be reconstituted by hepatocytes expanded in the one or more Fah-deficient mice. An analogous method could be used to selectively passage non-malignant cells from a patient suffering from a liver malignancy, such as HCC.

B. Fah-Deficient Mice as Liver Disease Models

Fah deficiency in animals leads to a disease phenotype similar to the human disease hereditary tyrosinemia type 1 (HT1). To prevent lethality, Fah-deficient animals are maintained on NTBC to prevent liver dysfunction (when the animals have not been repopulation with human hepatocytes that express FAH), however, titration of the dose of NTBC can be used promote the development of HT1-type phenotypes, including HCC, fibrosis and cirrhosis. In addition, immunodeficient and Fah-deficient mice repopulated with human hepatocytes can be induced to develop liver disease using, for example, a transforming agent, a toxic agent or by introducing malignant human hepatocytes. Accordingly, the Fah-deficient mice disclosed herein can be used to study a variety of liver diseases, including HCC and cirrhosis.

In some embodiments, the Fah-deficient mice disclosed herein are used as an animal model of human liver disease. Fah-deficient mice may be used as models of liver disease resulting from, for example, exposure to a toxin, infectious disease or malignancy or a genetic defect (i.e., Fah-deficiency leading to HT1). Examples of human genetic liver diseases for which Fah-deficient mice may serve as a model include, but are not limited to, hypercholesterolemia, hypertriglyceridemia, hyperoxaluria, phenylketonuria, maple syrup urine disease, glycogen storages diseases, lysosomal storage diseases (such as Gaucher's disease), and any inborn error of metabolism. The disclosed model systems can be used to gain a better understanding of particular liver diseases and to identify agents which may prevent, retard or reverse the disease processes.

In some cases, where the Fah-deficient mouse is to be used as a model for liver disease caused by a toxin, the Fah-deficient mouse is maintained on NTBC to prevent liver dysfunction until human hepatocyte repopulation in the liver is sufficient. The amount of toxic agent required to produce results most closely mimicking the corresponding human condition may be determined by using a number of Fah-deficient mice exposed to incremental doses of the toxic agent. Examples of toxic agents include, but are not limited to, ethanol, acetaminophen, phenyloin, methyldopa, isoniazid, carbon tetrachloride, yellow phosphorous and phalloidin. In some cases, the Fah-deficient mouse in the absence of human hepatocytes (but in the presence of NTBC or other similar compound) is used as the model for evaluating the effect of a toxin. In other examples, the Fah-deficient mouse is transplanted with human hepatocytes to evaluate the effect of the toxin on human hepatocytes. In these examples, it is not necessary to maintain the Fah-deficient mice on NTBC. Typically, expansion of human hepatocytes is allowed to proceed to the point where the size of the human hepatocyte population is substantial (e.g. has approached a maximum), before the Fah-deficient mouse is exposed to the toxic agent.

In some embodiments where a Fah-deficient mouse is to be used as a model for malignant liver disease (such as HCC or hepatoma) in the absence of human hepatocytes, the Fah-deficient mouse is administered a high enough dose of NTBC to prevent fatality due to liver dysfunction, but low enough to allow the development of HCC or other liver malignancy. Alternatively, the Fah-deficient mouse can be maintained on a dose of NTBC that prevents any liver dysfunction and the malignancy can be produced by exposure to a transforming agent or by the introduction of malignant cells. Another alternative is to transplant human hepatocytes and induce HCC, such as by using a transforming agent, or transplant the mice with malignant human hepatocytes. Thus, in some examples, the Fah-deficient mouse in the absence of human hepatocytes is used as the model for malignant liver disease. In other examples, the Fah-deficient mouse is transplanted with human hepatocytes to evaluate malignant liver disease of the human cells. In these examples, it is not necessary to maintain the Fah-deficient mice on NTBC. The transforming agent or malignant cells may be introduced with the initial colonizing introduction of human hepatocytes or after the human hepatocytes have begun to proliferate in the host animal. In the case of a transforming agent, it may be preferable to administer the agent at a time when human hepatocytes are actively proliferating.

Examples of transforming agents include aflatoxin, dimethylnitrosamine, and a choline-deficient diet containing 0.05-0.1% w/w DL-ethionine (Farber and Sarma, 1987, in Concepts and Theories in Carcinogenesis, Maskens et al., eds, Elsevier, Amsterdam, pp. 185-220). Such transforming agents may be administered either systemically to the animal or locally into the liver itself. Malignant cells may be inoculated directly into the liver.

C. Fah-Deficient Mice as Models for Hepatic Infection

Human hepatocytes expanded in and collected from Fah-deficient mice can also be used for a variety of microbiological studies. A number of pathogens (e.g., bacteria, viruses and parasites) will only replicate in a human host or in primary human hepatocytes. Thus, having a sufficient source of primary human hepatocytes is critical for studies of these pathogens. The expanded human hepatocytes can be used for studies of viral infection and replication or for studies to identify compounds that modulate infection of hepatic viruses. Methods of using primary human hepatocytes for studies of hepatic viruses are described in, for example, European Patent No. 1552740, U.S. Pat. No. 6,509,514 and PCT Publication No. WO 00/17338. Examples of hepatic viruses include hepatitis A virus, hepatitis B virus (HBV), hepatitis C virus (HCV) and cytomegalovirus (CMV). Examples of parasites that infect the liver include, for example, the causative agents of malaria (Plasmodium species, including Plasmodium falciparum, Plasmodium vivax, Plasmodium ovale, Plasmodium malariae and Plasmodium knowlesi) and the causative agents of leishmaniasis (Leishmania species, including L. donovani, L. infantum, L. chagasi, L. mexicana, L. amazonensis, L. venezuelensis, L. tropica; L. major; L. aethiopica, L. (V.) braziliensis, L. (V.) guyanensis, L. (V.) panamensis, and L. (V.) peruviana).

In addition to using the human hepatocytes expanded in Fah-deficient mice for microbiological studies, the Fah-deficient mice themselves can serve as animal models of hepatic pathogen infection. For example, Fah-deficient mice repopulated with human hepatocytes can be infected with a hepatic pathogen and used to screen candidate agents for treatment of the infection. Candidate agents include any compound from any one of a number of chemical classes, such as small organic compounds. Candidate agents also include biomolecules, such as, for example, nucleic acid molecules (including antisense oligonucleotides, small interfering RNAs, microRNAs, ribozymes, short hairpin RNAs, expression vectors and the like), peptides and antibodies, saccharides, fatty acids, steroids, purines, pyrimidines, derivatives, structural analogs or combinations thereof.

Candidate agents can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Using Fah-deficient mice to study HCV and HBV infection, as well as evaluate candidate agents for the treatment of these infections, is discussed below. However, the methods can be applied to any hepatic pathogen of interest. In one embodiment, a Fah-deficient mouse is used to identify agents that inhibit viral infection, decrease viral replication, and/or ameliorate one or more symptoms caused by HBV or HCV infection. In general, the candidate agent is administered to the Fah-deficient mouse, and the effects of the candidate agent assessed relative to a control. For example, the candidate agent can be administered to an HCV-infected Fah-deficient mouse, and the viral titer of the treated animal (e.g., as measured by RT-PCR of serum samples) can be compared to the viral titer of the animal prior to treatment and/or to an untreated HCV-infected animal. A detectable decrease in viral titer of an infected animal following treatment with a candidate agent is indicative of antiviral activity of the agent.

The candidate agent can be administered in any suitable manner appropriate for delivery of the agent. For example, the candidate agent can be administered by injection (such as by injection intravenously, intramuscularly, subcutaneously, or directly into the target tissue), orally, or by any other desirable means. In some cases, the in vivo screen will involve a number of Fah-deficient mice receiving varying amounts and concentrations of the candidate agent (from no agent to an amount of agent that approaches an upper limit of the amount that can be safely delivered to the animal), and may include delivery of the agent in different formulations and routes. Candidate agents can be administered singly or in combinations of two or more, especially where administration of a combination of agents may result in a synergistic effect.

The activity of the candidate agent can be assessed using any one of a variety of means known in the art. For example, where the Fah-deficient mouse is infected with a hepatotropic pathogen (e.g., HBV or HCV), the effect of the agent can be assessed by examining serum samples for the presence of the pathogen (e.g., measuring viral titer) or markers associated with the presence of the pathogen (e.g., a pathogen-specific protein or encoding nucleic acid). Qualitative and quantitative methods for detecting and assessing the presence and severity of viral infection are well known in the art. In one embodiment, the activity of an agent against HBV infection can be assessed by examining serum samples and/or tissue sections for the presence of a viral antigen (such as HBV surface antigen (HBsAg) or HBV core antigen (HbcAg)). In another embodiment, the activity of an agent against viral infection can be assessed by examining serum samples for the presence of viral nucleic acid (such as HCV RNA). For example, HCV RNA can be detected using, for example, reverse transcriptase polymerase chain reaction (RT-PCR), competitive RT-PCR or branched-DNA (bDNA) assay, detection of negative-strand RNA (the replicative intermediate of HCV) by RT-PCR, or sequencing of viral RNA to detect mutation/shift in the viral genome (“quasispecies evolution”) with therapy. Alternatively or in addition, the host liver may be biopsied and in situ RT-PCR hybridization performed to demonstrate directly any qualitative or quantitative alterations in the amount of viral particles within tissue sections. Alternatively or in addition, the host can be euthanized and the liver examined histologically for signs of infection and/or toxicity caused by the agent.

Fah-deficient mice can also be used to screen candidate vaccines for their ability to prevent or ameliorate infection by a hepatotropic pathogen. In general, a vaccine is an agent that, following administration, facilitates the host in mounting an immune response against the target pathogen. The humoral, cellular, or humoral/cellular immune response elicited can facilitate inhibition of infection by the pathogen against which the vaccine is developed. Of particular interest in the present disclosure are vaccines that elicit an immune response that inhibits infection by and/or intrahepatic replication of a hepatotropic pathogen (e.g., a microbial, viral, or parasitic pathogen), particularly a viral pathogen, such as HBV and/or HCV.

To evaluate candidate vaccines, the Fah-deficient mice are transplanted with human hepatocytes to repopulate the mouse liver with human hepatocytes. Screening for an effective vaccine is similar to the screening methods described above. In some embodiments, the candidate vaccine is administered to the Fah-deficient mouse prior to inoculation with the hepatotropic pathogen. In some cases, the candidate vaccine is administered by providing a single bolus (e.g., intraperitoneal or intramuscular injection, topical administration, or oral administration), which is optionally followed by one or more booster immunizations. The induction of an immune response can be assessed by examining B and T cell responses that are specific for the antigen/vaccine according to methods well known in the art. The immunized Fah-deficient mouse is then challenged with the hepatotropic pathogen. Typically, several immunized animals are challenged with increasing titers of the pathogen. The animals are then observed for development of infection, and the severity of infection is assessed (such as by assessing the titer of the pathogen present, or examining human hepatocyte function parameters). Vaccine candidates that provide for a significant decrease in infection by the pathogen and/or a significant decrease in the severity of disease that results post-challenge are identified as viable vaccines.

D. Pharmacology, Toxicology and Gene Therapy Studies

Fah-deficient mice and/or human hepatocytes expanded in and collected from Fah-deficient mice can be used to evaluate alterations in gene expression in human hepatocytes by any pharmacologic compound, such as small molecules, biologicals, environmental or biological toxins or gene delivery systems.

As described in the Examples below, expression of genes involved in drug conjugation and detoxification, including several of the hepatocyte transporter proteins, was detected in expanded human hepatocytes collected from recipient mice. Recent studies have shown the critical role played by these conjugation pathways (Kostrubsky et al. Drug. Metab. Dispos. 28:1192-1197, 2000) and hepatocyte transporter proteins (Kostrubsky et al. Toxicol. Sci. 90:451-459, 2006) in predicting drug toxicity. Along with a normal human response to CYP induction by exogenous drugs, such as rifampicin or PB, or BNF, the expression of the nuclear hormone receptor transcription factors, the conjugation pathways and major transport proteins by the human hepatocytes expanded in FRG mice allow for the assessment of the role of these gene products in human drug metabolism and toxicity, in vivo.

Humanized FRG mice display blood lipid profiles typical of a human with a significantly higher content of LDL cholesterol (see Table 4), whereas control mice display the typical high HDL content of mouse blood. Humanized FRG mice will therefore be useful to test anti-hyperlipidemia drugs and anti-atherosclerosis medications. Humanized FRG mice display a human bile acid composition in the gall bladder (see Table 3). This indicates that these mice can be used to assay biliary excretion of normal and xenobiotic metabolites.

For example, human hepatocytes expanded in and collected from Fah-deficient mice can be used to evaluate toxicity of particular compounds in human cells. Methods of testing toxicity of compounds in isolated hepatocytes are well known in the art and are described, for example, in PCT Publication No. WO 2007/022419. Similarly, Fah-deficient mice transplanted with human hepatocytes can be used to evaluate the toxicity of exogenous agents. In some embodiments, the exogenous agent is a known or suspected toxin.

In some embodiments, Fah-deficient mice transplanted with human hepatocytes (or human hepatocytes expanded in and collected from Fah-deficient mice) are used to evaluate any one of a number of parameters of drug metabolism and pharmacokinetics. For example, studies can be carried out to evaluate drug metabolism, drug/drug interactions in vivo, drug half-life, routes of excretion/elimination, metabolites in the urine, feces, bile, blood or other bodily fluid, cytochrome p450 induction, enterohepatic recirculation, and enzyme/transporter induction.

In some embodiments, Fah-deficient mice transplanted with human hepatocytes (or human hepatocytes expanded in and collected from Fah-deficient mice) are used to evaluate toxicology and safety of a compound, including therapeutic agents or candidate agents (such as small molecules or biologicals), environmental or biological toxins, or gene delivery systems. For example, cell cycle proliferation in human hepatocytes can be evaluated, such as to determine the risk of cancer following exposure to the compound. Toxicity to hepatocytes can also be assessed, such as by histology, apoptosis index, liver function tests and the like. Analysis of hepatocyte metabolism can also be performed, such as analysis of metabolites after infection of stable isotope precursors.

The efficacy of particular drugs can also be evaluated in Fah-deficient mice transplanted with human hepatocytes. Such drugs include, for example, drugs to treat hyperlipidemia/atherosclerosis, hepatitis and malaria.

In some embodiments, Fah-deficient mice transplanted with human hepatocytes (or human hepatocytes expanded in and collected from Fah-deficient mice) are used to study gene therapy protocols and vectors. For example, the following parameters can be evaluated: transduction efficiency of gene delivery vehicles including viral and non-viral vectors; integration frequency and location of genetic payloads (integration site analysis); functionality of genetic payloads (gene expression levels, gene knockdown efficiency); and side effects of genetic payloads (analysis of gene expression or proteomics in human hepatocytes in vivo). For example, use of transfected hepatocytes in gene therapy of a patient suffering from familial hypercholesterolemia has been reported (Grossman et al. Nat. Genet. 6: 335, 1994).

### VIII. Vectors Encoding Urokinase

In some embodiments of the methods described herein, Fah-deficient mice are administered a vector encoding urokinase prior to transplantation of human hepatocytes. In one embodiment, the urokinase (also known as urokinase plasminogen activator (uPA)) is the secreted form of human urokinase. In another embodiment, the urokinase is a modified, non-secreted form of urokinase (see U.S. Pat. No. 5,980,886). Any type of suitable vector for expression of urokinase in mice is contemplated. Such vectors include plasmid vectors or viral vectors. Suitable vectors include, but are not limited to, DNA vectors, adenovirus vectors, retroviral vectors, pseudotyped retroviral vectors, AAV vectors, gibbon ape leukemia vector, VSV-G, VL30 vectors, liposome mediated vectors, and the like. In one embodiment, the viral vector is an adenovirus vector. The adenovirus vector can be derived from any suitable adenovirus, including any adenovirus serotype (such as, but not limited to Ad2 and Ad5). Adenovirus vectors can be first, second, third and/or fourth generation adenoviral vectors or gutless adenoviral vectors. The non-viral vectors can be constituted by plasmids, phospholipids, liposomes (cationic and anionic) of different structures. In another embodiment, the viral vector is an AAV vector. The AAV vector can be any suitable AAV vector known in the art.

Viral and non-viral vectors encoding urokinase are well known in the art. For example, an adenovirus vector encoding human urokinase is described in U.S. Pat. No. 5,980,886 and by Lieber et al. (Proc. Natl. Acad. Sci. U.S.A. 92(13):6210-4, 1995). U.S. Patent Application Publication No. 2005-176129 and U.S. Pat. No. 5,585,362 describe recombinant adenovirus vectors and U.S. Pat. No. 6,025,195 discloses an adenovirus vector for liver-specific expression. U.S. Patent Application Publication No. 2003-0166284 describes adeno-associated virus (AAV) vectors for liver-specific expression of a gene of interest, including urokinase. U.S. Pat. Nos. 6,521,225 and 5,589,377 describe recombinant AAV vectors. PCT Publication No. WO 0244393 describes viral and non-viral vectors comprising the human urokinase plasminogen activator gene. An expression vector capable of high level of expression of the human urokinase gene is disclosed in PCT Publication No. WO 03087393.

Vectors encoding urokinase can optionally include expression control sequences, including appropriate promoters, enhancers, transcription terminators, a start codon (i.e., ATG) in front of a protein-encoding gene, splicing signal for introns and maintenance of the correct reading frame of that gene to permit proper translation of mRNA, and stop codons. Generally expression control sequences include a promoter, a minimal sequence sufficient to direct transcription.

The expression vector can contain an origin of replication, a promoter, as well as specific genes which allow phenotypic selection of the transformed cells (such as an antibiotic resistance cassette). Generally, the expression vector will include a promoter. The promoter can be inducible or constitutive. The promoter can be tissue specific. Suitable promoters include the thymidine kinase promoter (TK), metallothionein I, polyhedron, neuron specific enolase, thyrosine hyroxylase, beta-actin, or other promoters. In one embodiment, the promoter is a heterologous promoter.

In one example, the sequence encoding urokinase is located downstream of the desired promoter. Optionally, an enhancer element is also included, and can generally be located anywhere on the vector and still have an enhancing effect. However, the amount of increased activity will generally diminish with distance.

The vector encoding urokinase can be administered by a variety of routes, including, but not limited to, intravenously, intraperitoneally or by intravascular infusion via portal vein. The amount of vector administered varies and can be determined using routine experimentation. In one embodiment, FRG mice are administered an adenovirus vector encoding urokinase at a dose of about 1×10⁸ to about 1×10¹⁰ plaque forming units. In one preferred embodiment, the dose is about 5×10⁹ plaque forming units.

In one exemplary embodiment, FRG mice are administered an adenovirus vector encoding human urokinase. Adenovirus vectors have several advantages over other types of viral vectors, such as they can be generated to very high titers of infectious particles; they infect a great variety of cells; they efficiently transfer genes to cells that are not dividing; and they are seldom integrated in the guest genome, which avoids the risk of cellular transformation by insertional mutagenesis (Douglas and Curiel, Science and Medicine, March/April 1997, pages 44-53; Zern and Kresinam, Hepatology:25(2), 484-491, 1997). Representative adenoviral vectors which can be used to encode urokinase are described by Stratford-Perricaudet et al. (J. Clin. Invest. 90: 626-630, 1992); Graham and Prevec (In Methods in Molecular Biology: Gene Transfer and Expression Protocols 7: 109-128, 1991); and Barr et al. (Gene Therapy, 2:151-155, 1995).

The following examples are provided to illustrate certain particular features and/or embodiments. These examples should not be construed to limit the disclosure to the particular features or embodiments described.

### EXAMPLES

### Example 1

### Generation of Fah^{-/-}/Rag2^{-/-}/Il2rg^{-/-} (FRG) Mice

A number of different strategies can be employed to produce an immunodeficient mouse, including, for example, by administration of an immunosuppressive drug, or by introducing one or more genetic alterations. This example describes the generation of a genetically altered immunodeficient mouse.

To generate an immunodeficient Fah^{-/-} mouse strain completely lacking T cells, B cells and NK cells, but without a DNA repair defect, Fah^{-/-}/Rag2^{-/-}/Il2rg^{-/-} (FRG) mice were generated. Male Fah^{-/-}129S4 mice (Grompe et al. Genes Dev. 7:2298-2307, 1993) were crossed with female Rag2^{-/-}/Il2rg^{-/-} mice (Taconic). All animals were maintained with drinking water containing 2-(2-nitro-4-trifluoro-methyl-benzoyl)-1,3 cyclohexanedione (NTBC) at a concentration of 1.6 mg/L (Grompe et al. Nat. Genet. 10:453-460, 1995). To confirm the genotypes of each animal, PCR-based genotyping was carried out on 200 ng genomic DNA isolated from toe tissue (Grompe et al. Genes Dev. 7:2298-2307, 1993; Traggiai et al. Science 304:104-107, 2004).

FRG mice grew well and were fully fertile if they were continuously given NTBC in their drinking water. FRG mouse livers were macroscopically normal in size and shape, and histological examination showed no differences between conventional Fah^{-/-} mice and the FRG mice. As in conventional Fah^{-/-} mice, NTBC withdrawal resulted in gradual hepatocellular injury in FRG mice and eventual death after 4-8 weeks (Overturf et al. Nat. Genet. 12:266-273, 1996).

### Example 2

### Histology and Engraftment Detection Assays

### Histology and Immunocytochemistry

FAH immunohistochemistry was performed as previously described (Wang et al. Am. J. Pathol. 161:565-574, 2002). Briefly, liver and kidney tissues fixed in 10% phosphate-buffered formalin, pH 7.4, were dehydrated in 100% ethanol and embedded in paraffin wax at 58° C. Deparaffinized 4-μm sections were stained with hematoxylin and eosin. For immunohistochemistry, sections were treated with 3% H₂O₂ in methanol for endogenous peroxydase blocking Avidin and biotin blocking was also performed before incubation with primary antibodies. Sections were incubated with anti-FAH rabbit antibody or HepPar antibody (DAKO) for 2 hours at room temperature followed by HRP-conjugated secondary antibody incubation. Signals were detected by diaminobenzidine (DAB).

### FAH Enzyme Assay

Fumarylacetoacetate was incubated with cytosolic liver fractions from recipient liver, and disappearance speed was measured spectroscopically at 330 nm. Wild type and Fah^{-/-} livers were used as positive and negative control respectively. Fumarylacetoacetate was prepared enzymatically from homogentisic acid (Knox et al. Methods Enzymol. 2:287-300, 1955).

### Genomic PCR for Alu Sequence

Genomic DNA was isolated from the liver using the DNeasy tissue kit (Qiagen). Human Alu sequences were amplified by PCR according to standard procedures with the following primers 5′-GGCGCGGTGGCTCACG-3′ (SEQ ID NO: 1) and 5′-TTTTTTGAGACGGAGTCTCGCTC-3′ (SEQ ID NO: 2).

### RT-PCR for Hepatocyte Specific Gene Expression

Total RNA was isolated from the liver using the RNeasy mini kit (Qiagen). Complementary DNA was synthesized by reverse transcriptase with an oligo-dT primer. The primers shown in Table 1 were used for human or mouse specific cDNA amplification.

TABLE 1
RT-PCR Primers for Amplification of Hepatocyte Specific Genes

| Primer Sequence | Primer Description | SEQ ID NO: |
|---|---|---|
| ATGGATGATTTCGCAGCTTT | human ALB forward | 3 |
| TGGCTTTACACCAACGAAAA | human ALB reverse | 4 |
| TACAGCGGAGCAACTGAAGA | mouse Alb forward | 5 |
| TTGCAGCACAGAGACAAGAA | mouse Alb reverse | 6 |
| CCGGGAGAGTTTTACCACAA | human TAT forward | 7 |
| CCTTCCCTAGATGGGACACA | human TAT reverse | 8 |
| CTGACCTCACCTGGGACAAT | human TF forward | 9 |
| CCTCCACAGGTTTCCTGGTA | human TF reverse | 10 |
| TTTGGGACCACTGTCTCTCC | human FAH forward | 11 |
| CTGACCATTCCCCAGGTCTA | human FAH reverse | 12 |
| ATGGCTTCTCATCGTCTGCT | human TTR forward | 13 |
| GCTCCTCATTCCTTGGGATT | human TTR reverse | 14 |
| GTGCCTTTATCACCCATGCT | human UGT1A1 forward | 15 |
| TCTTGGATTTGTGGGCTTTC | human UGT1A1 reverse | 16 |

### Human Albumin Measurement

Small amounts of blood were collected once a week from the left saphenous vein with a heparinized blood capillary. After 1,000 or 10,000× dilution with Tris-buffered saline, human albumin concentration was measured with the Human Albumin ELISA Quantitation Kit (Bethyl) according to the manufacturer's protocol.

### Fluorescent Immunocytochemistry

Hepatocytes from humanized mouse livers were suspended in Dulbecco's modified Eagle's medium (DMEM) and plated on collagen typel-coated 6-well plates. Attached cells were fixed with 4% paraformaldehyde for 15 minutes and blocked with 5% skim milk. Rabbit anti-FAH, goat anti-human albumin (Bethyl), goat anti-mouse albumin (Bethyl) were used as primary antibodies at dilution of 1/200. ALEXA™ Fluoro 488 anti-goat IgG (Invitrogen) or ALEXA™ Fluoro 555 anti-rabbit IgG (Invitrogen) were used as secondary antibody. The images were captured with an AXIOVERT™ 200 microscope by using Nikon digital camera.

### FACS Analysis

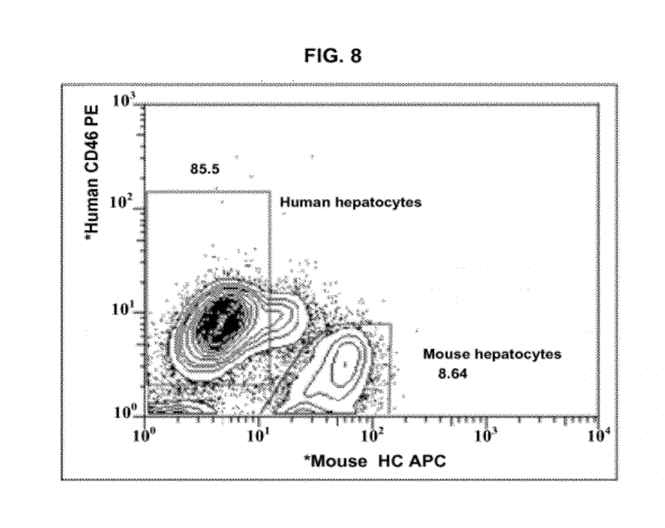

After dissociation of the recipient livers, parenchymal cells were incubated at 4° C. for 30 minutes with fluorescein isothiocyanate (FITC)-conjugated anti-human human leukocyte antigen (HLA)-A,B,C (BD Pharmingen) and phycoerythrin (PE)-conjugated anti-mouse H2-K(b) (BD Pharmingen) antibodies. They were then rinsed with PBS twice and analyzed with a FACS CALIBUR™ (Becton Dickinson) flow cytometer. FITC-conjugated and PE-conjugated IgG were used as negative controls (see FIG. 8).

### Fluorescence In Situ Hybridization

Total genomic DNA probes were generated by nick translation of total mouse and human genomic DNA. Cy3-dUTP incorporation was carried out according to manufacturer's recommendations (Invitrogen). Final probe concentration was 200 ng/μl. Slides with attached cells were treated with RNase at 100 mg/ml for 1 hour at 37° C. and washed in 2×SSC for three 3-minute rinses. Following wash steps, slides were dehydrated in 70, 90 and 100% ethanol for 3 min each. Chromosomes were denatured at 75° C. for 3 minutes in 70% formamide/2×SSC, followed by dehydration in ice cold 70%, 90% and 100% ethanol for 3 minutes each. Probe cocktails were denatured at 75° C. for 10 minutes and pre-hybridized at 37° C. for 30 minutes. Probes were applied to slides and incubated overnight at 37° C. in a humid chamber. Post-hybridization washes consisted of three 3-minute rinses in 50% formamide/2×SSC and three 3-minute rinses in PN buffer (0.1 M Na2HPO4, 0.1 M NaH2PO4, pH 8.0, 2.5% NONIDET™ P-40), all at 45° C. Slides were then counterstained with Hoechst (0.2 ug/ml), cover-slipped and viewed under UV fluorescence (Zeiss).

### Example 3

### Isolation and Cryopreservation of Human Hepatocytes

Human hepatocytes were isolated from donor livers that were not used for liver transplantation according to previously described procedures (Strom et al. Cell Transplant. 15:S105-110, 2006). Briefly, liver tissue was perfused with calcium and magnesium-free Hanks' balanced salt solution (Cambrex) supplemented with 0.5 mM EGTA (Sigma) and HEPES (Cellgro), followed by digestion with 100 mg/L collagenase XI (Sigma) and 50 mg/L deoxyribonuclease I (Sigma) in Eagle's minimal essential medium (Cambrex) through the existing vasculature. The cells were washed three times with Eagle's minimal essential medium plus 7% bovine calf serum (Hyclone) at 50×g for 2 minutes. Pelleted hepatocytes were transferred into cold VIASPAN™ (a universal aortic flush and cold storage solution for the preservation of intra-abdominal organs; also referred to as University of Wisconsin solution, or UW).

Shipped hepatocytes were transferred into VIASPAN™ solution supplemented with 10% fetal bovine serum and 10% dimethylsulfoxide at 5×10⁶ hepatocytes per ml. The cryotubes were thickly wrapped with paper towels, stored at −80° C. for one day and finally transferred into liquid nitrogen. For thawing, cells were rapidly reheated in a 37° C. water bath and DMEM was added gradually to minimize the speed of change of the DMSO concentration.

### Example 4

### Repopulation of FRG Mouse Liver with Human Hepatocytes

Overexpression of urokinase has been shown to enhance hepatocyte engraftment in several systems (Lieber et al. Hum. Gene Ther. 6:1029-1037, 1995). Therefore, experiments were performed to determine whether administration of a urokinase expressing adenovirus prior to transplantation of human hepatocytes would be beneficial. The adenoviral vector expressing the secreted form of human urokinase (urokinase plasminogen activator; uPA) has been previously described (Lieber et al. Proc. Natl. Acad. Sci. U.S.A. 92:6210-6214, 1995 and U.S. Pat. No. 5,980,886).

Donor hepatocytes were isolated and transplanted 24-36 hours after isolation. In the majority of cases, the cells were preserved in VIASPAN™ solution and kept at 4° C. during transport. However, in two experiments, cryopreserved hepatocytes were transplanted. The viability and quality of donor hepatocytes was highly variable with plating efficiencies ranging from 10% to 60%.

For transplantation, the following general protocol was used. Adult (6 to 15 week old) male or female FRG mice were given an intravenous injection (retroorbital) of uPA adenovirus (5×10⁹ plaque forming units (PFU) per mouse) 24-48 hours before transplantation. One million viable human hepatocytes (determined by trypan blue exclusion) in 100 μl of Dulbecco's modified essential medium were injected intrasplenically via a 27 gauge needle. NTBC was gradually withdrawn over the next six days (1.6 mg/L day 0-2; 0.8 mg/L day 3-4; 0.4 mg/L day 5-6) and completely withdrawn one week after transplantation. Two weeks after stopping NTBC, the animals were put back on the drug for five days and then taken off again.

In three separate transplantations, primary engraftment of human hepatocytes was observed in FRG mice in recipients which had first received the uPA adenovirus. The uPA-pretreatment regimen was therefore used in most subsequent transplantation experiments.

In total, human hepatocytes from nine different donors were used successfully and no engraftment failures occurred after introduction of the uPA adenovirus regimen. Of these, seven were isolated from the livers of brain-dead organ donors and two were isolated from surgical liver resections. Donor ages varied from 1.2 to 64 years (Table 2).

TABLE 2
Summary of Engraftment Results from each Hepatocyte Donor

| | | Age | Number of mice | Human albumin |
|---|---|---|---|---|
| Donor | Origin | (years) | transplanted | positive (%) |
| A | cadaveric | 1.8 | 6 | N/A |
| B | resection | 55 | 9 | 3 (33) |
| C | resection | 50 | 5 | 1 (20) |
| D | cadaveric | 1.2 | 2 | 1 (50) |
| E | cadaveric | 55 | 5 | 2 (40) |
| | (cryopreserved) | | | |
| G | commercial | N/A | 8 | 1 (13) |
| | (cryopreserved) | | | |
| H | cadaveric | 64 | 6 | 2 (33) |
| I | cadaveric | 59 | 5 | 3 (60) |
| J | cadaveric | 1.3 | 6 | 4 (60) |

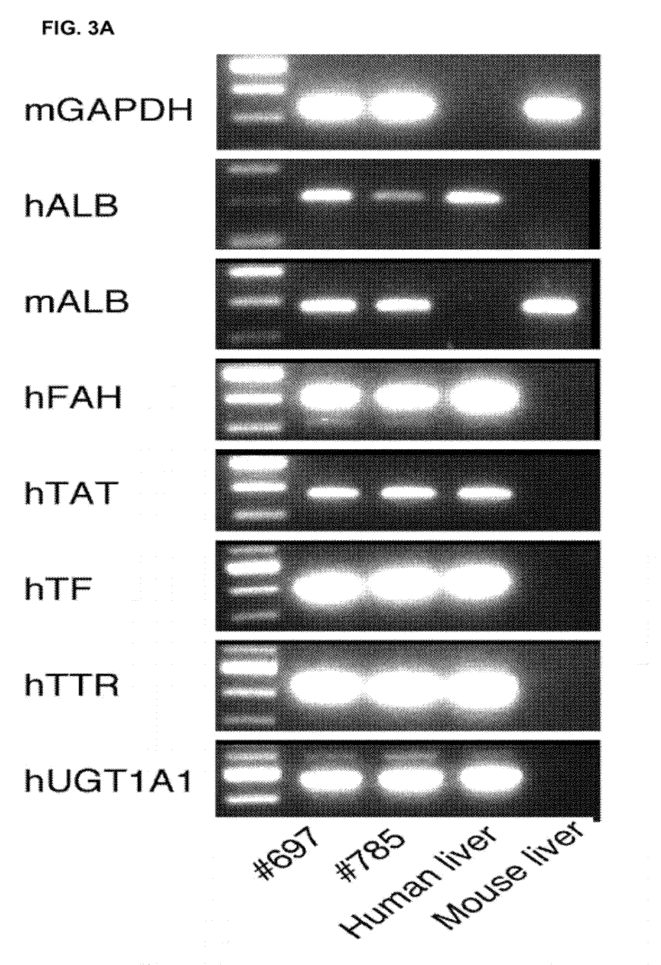

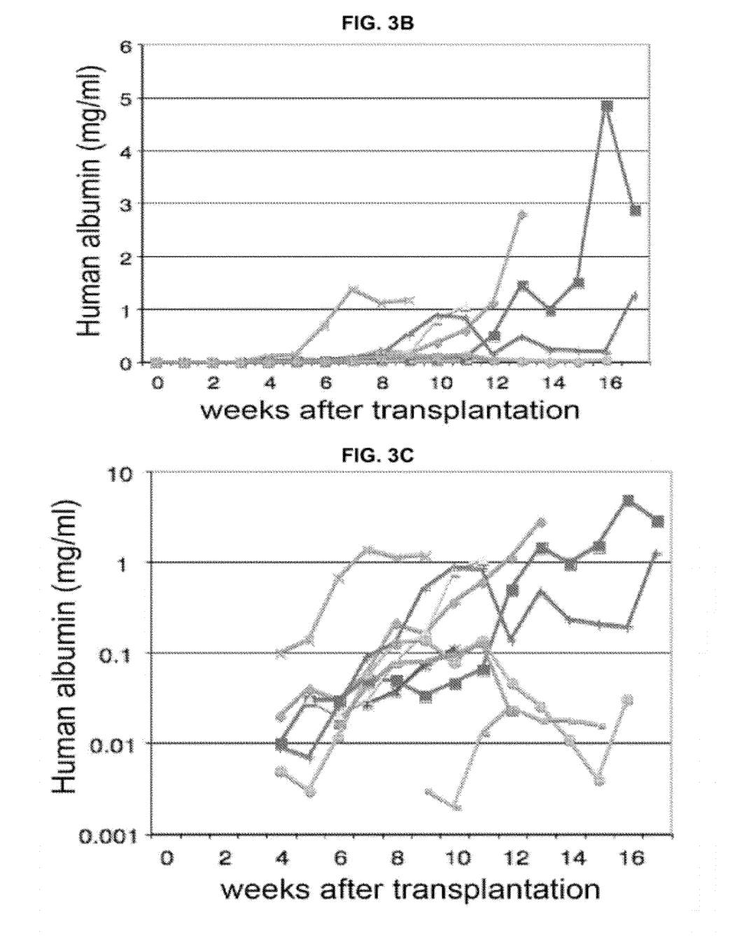

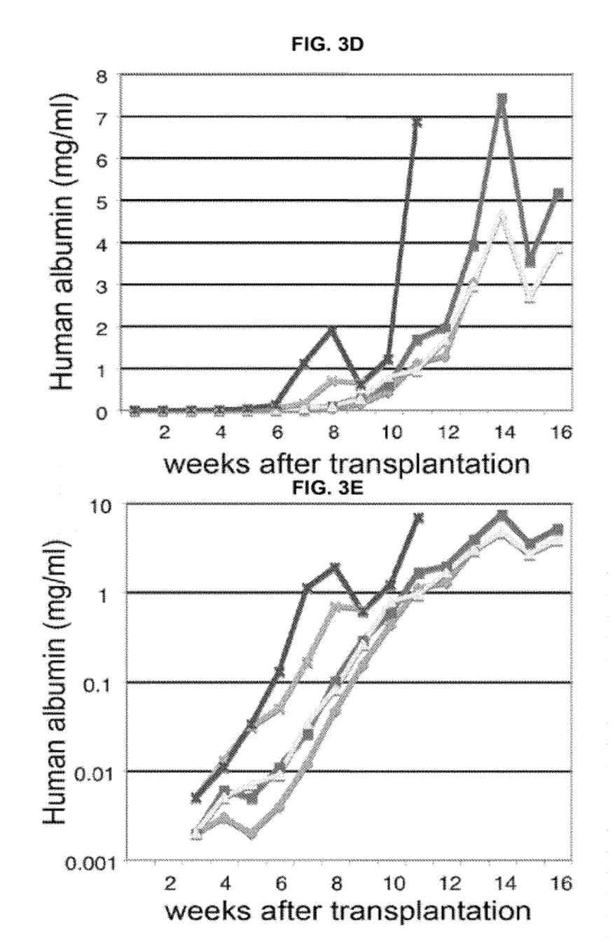

In all experiments, at least one recipient became significantly engrafted (>1% human cells) with human hepatocytes using this protocol, regardless of the cell batch used. Engraftment was demonstrated by different methods including histology, DNA analysis, enzyme assay and in later experiments, human serum albumin. In the transplantations monitored by albumin levels, 17 of 43 (39.5%; range 12 to 67%) primary recipients became repopulated (Table 2 and FIG. 3). Of these, seven were highly repopulated (30-90%) and achieved albumin levels >1 mg/ml. Not only hepatocytes from cadaveric livers, but also from hepatic resections, were engrafted. Furthermore, cryopreserved cells were also successfully engrafted.

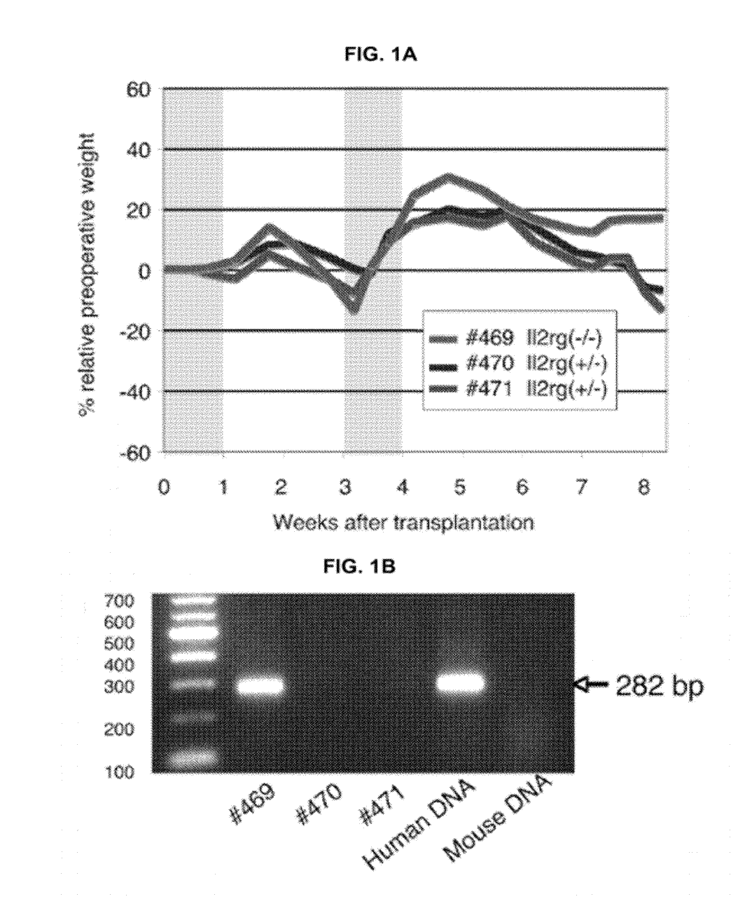

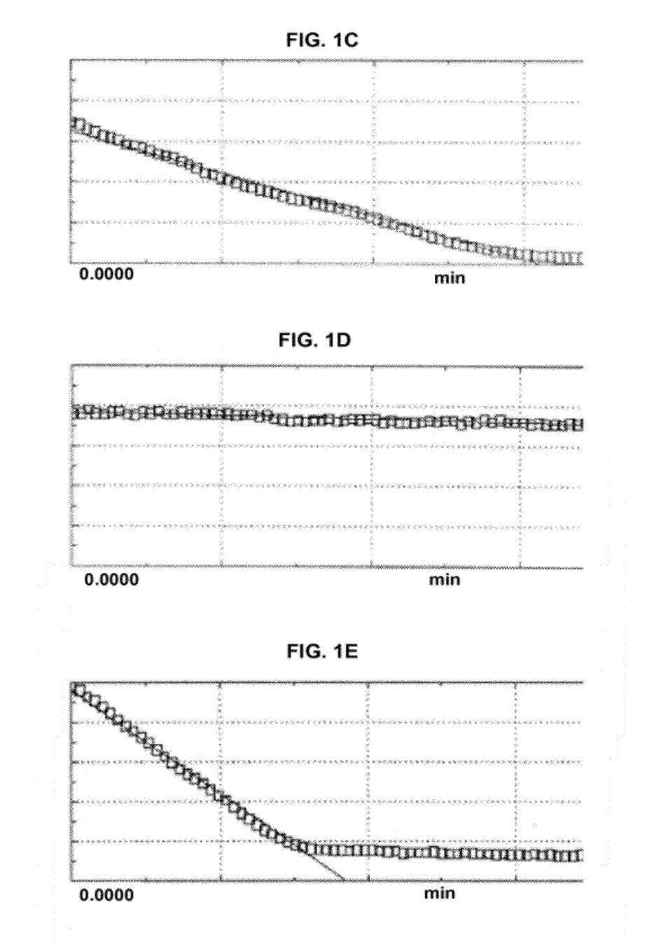

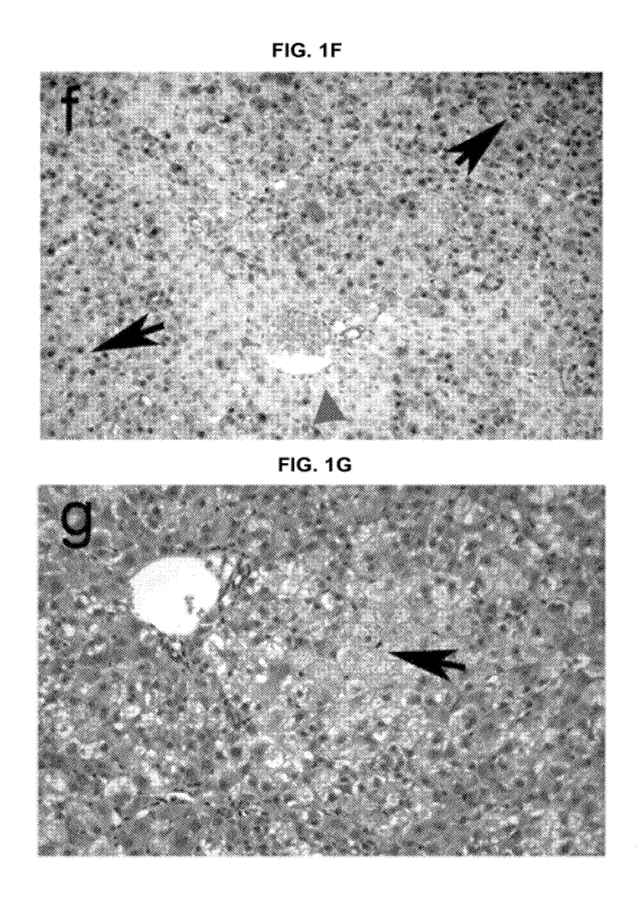

In highly engrafted mice (>30% repopulation), the weight of transplanted FRG mice stabilized during the second NTBC withdrawal, whereas fewer immune deficient litter mates heterozygous for Il2rg (Fah^{-/-}/Rag2^{-/-}/Il2rg^{-/-}) given the same cells continued to lose weight (FIG. 1A). This weight stabilization in triple mutant mice suggested that the transplanted human hepatocytes were replacing the functions of the diseased Fah^{-/-} recipient hepatocytes. Upon complete weight stabilization (2-3 months after initial transplantation), the recipient livers were then harvested. Macroscopically, FRG livers were normal in shape and weight and without macroscopic nodules. Genomic PCR for human-specific Alu DNA-sequences was positive in FRG recipient livers, whereas Il2rg heterozygotes were all negative (FIG. 1B). To directly confirm hepatocytic function of the repopulating cells, FAH enzyme activity was assayed (Knox et al. Methods Enzymol. 2:287-300, 1955).

Recipient mouse livers had considerable amounts of FAH enzyme activity, equaling or exceeding normal mouse liver (FIGS. 1C-1E). As FAH is expressed exclusively in fully differentiated hepatocytes, this suggested the transplanted human hepatocytes were not dedifferentiated or abnormal when engrafted in mouse liver. FAH immunostaining confirmed that more than 70% of liver parenchyma was repopulated with FAH-positive human hepatocytes (FIG. 1F and FIG. 1G).

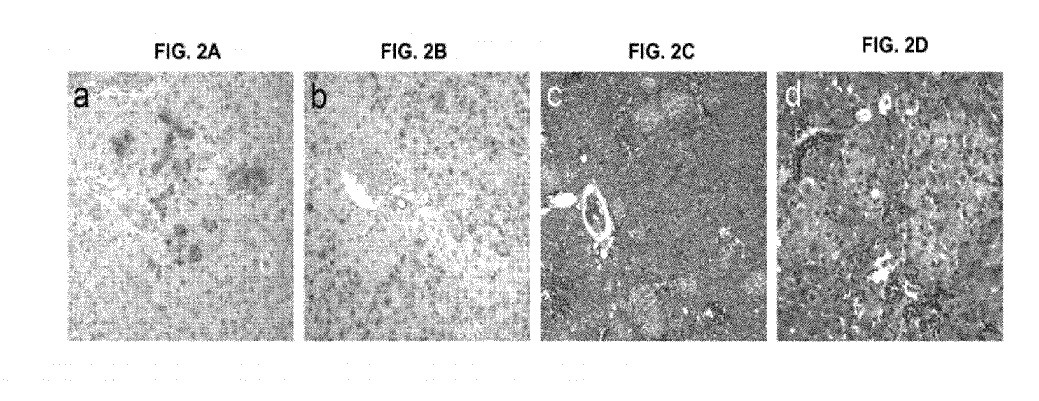

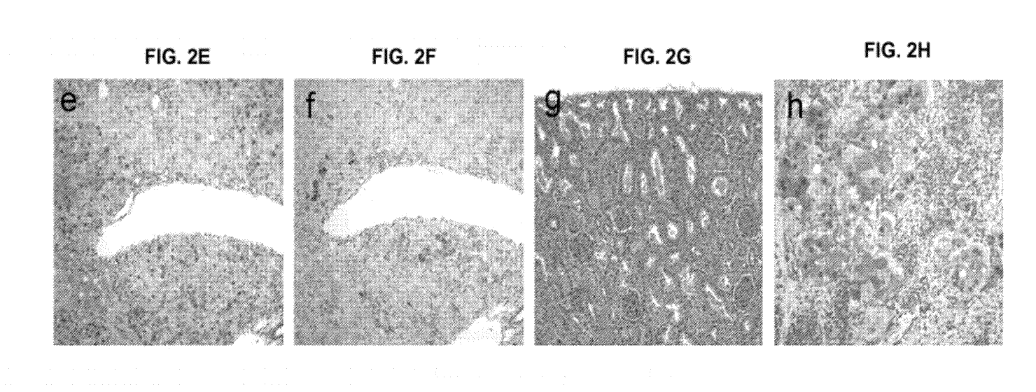

Histological and immunohistochemical examination was performed using additional recipient livers (FIG. 2A and FIG. 2B). FAH-positive human hepatocytes appeared completely integrated into the structure of the recipient liver. In several recipients, the engrafted hepatocytes occupied more than of 80% parenchyma without disturbing the recipient liver organization (FIGS. 2B, 2E and 2F). Clonally expanding human hepatocytes could be clearly distinguished from mouse hepatocytes morphologically, by size, and by their pale cytoplasm (FIG. 2C and FIG. 2D). The size of human hepatocytes was relatively large, and their cytoplasm looked bright, probably because of glycogen accumulation as previously reported (Meuleman et al. Hepatology 41:847-856, 2005). FAH-positive hepatocytes were also positive for HepPar antibody, which specifically labels human hepatocytes but not mouse counterparts (FIG. 2E and FIG. 2F). In contrast, the FAH-negative areas displayed necroinflammation and contained dysplastic hepatocytes consistent with the findings in conventional Fah^{-/-} mice after NTBC withdrawal.

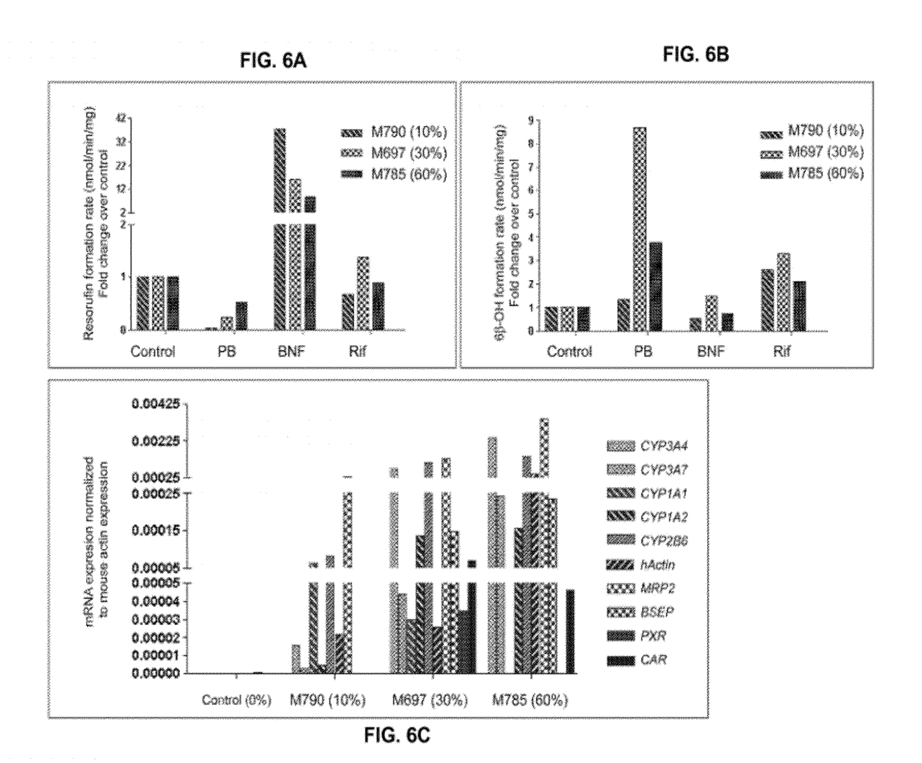

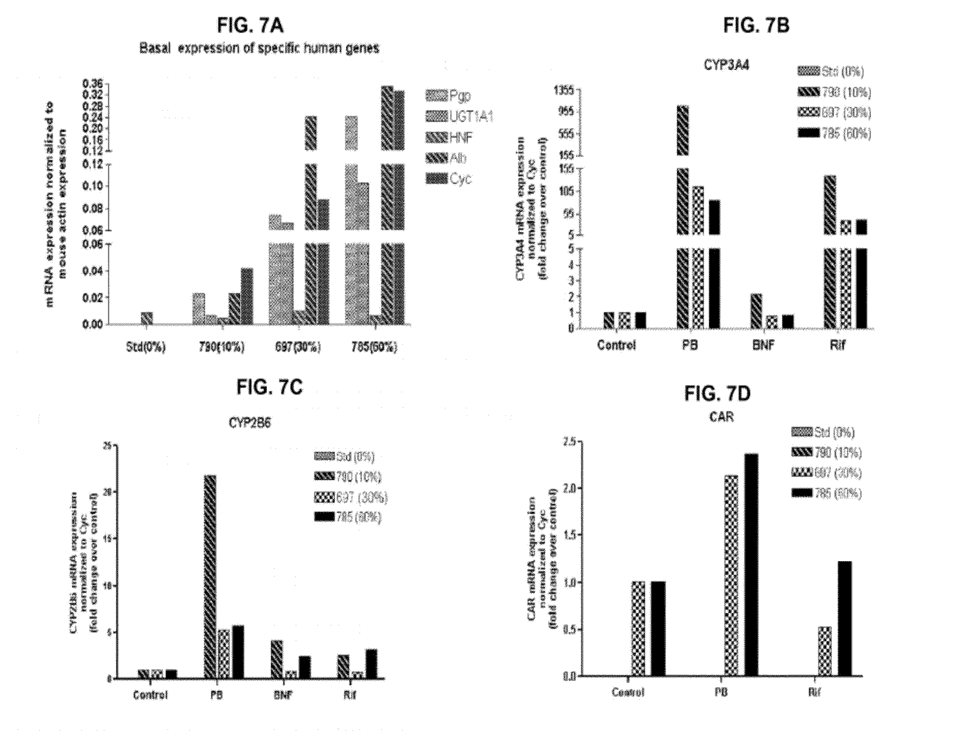

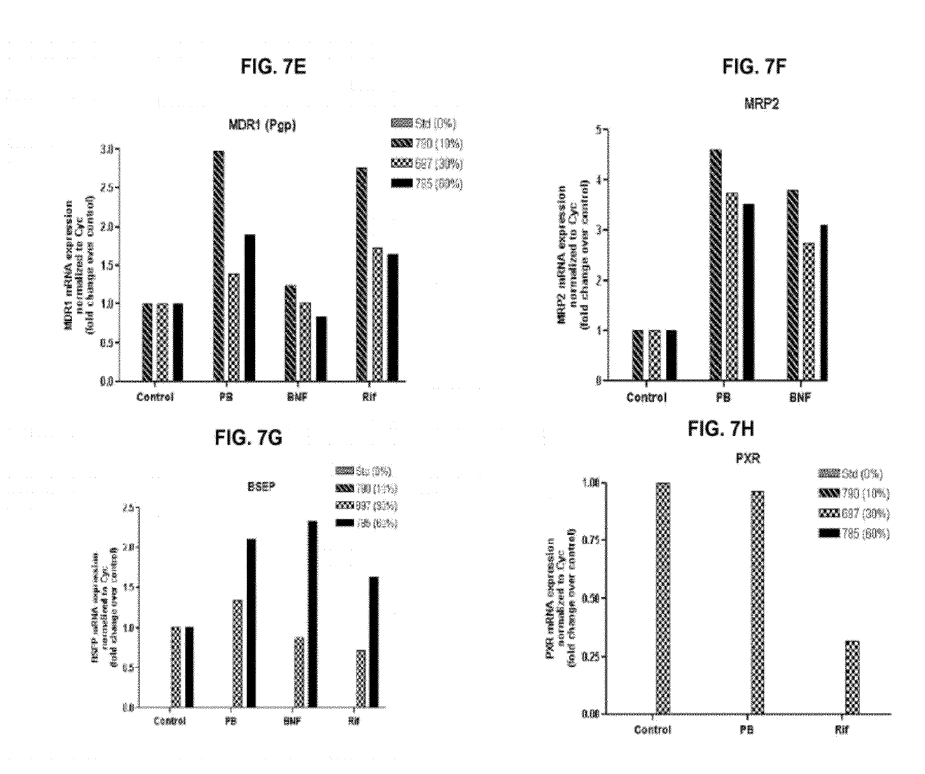

To examine whether repopulated human hepatocytes expressed mature hepatocyte-specific genes, RT-PCR was performed on messenger RNA extracted from recipient livers. The human albumin (ALB), FAH, transferrin (TF), transthyretin (TTR), tyrosine aminotransferase (TAT), and UGT1A1 genes were abundantly expressed in recipient livers (FIG. 3A, FIG. 6C and FIG. 7). Hepatocyte functionality was also assessed by measuring blood concentration of human albumin. An ELISA kit specific for human albumin was used, and the threshold for detection was 0.05 μg/ml using samples diluted 1:100. Human albumin was first detected at 4 to 10 weeks after transplantation in primary recipients. Although initially there was some fluctuation in levels, concentrations then increased relatively steadily for several more weeks (FIG. 3B and FIG. 3C).

To further evaluate the functionality of human hepatocytes in chimeric mice, the bile acid composition and lipoprotein profiles of humanized mice were compared to normal human and mouse bile and lipoprotein profiles. As shown below, the bile of mice with humanized levels resembles human bile in its composition (Table 3) and humanized mice have high LDL and cholesterol levels similar to those of humans (Table 4). The presence of deoxycholic acid is typical of human bile and the levels of β-muricholic acid were much lower than in normal mouse bile. Cholesterol levels were much higher in humanized mice and this increase could be attributed to LDL cholesterol.

TABLE 3
Bile acid analysis of repopulated mice

| Mouse | DCA | CDCA/α-muri | CA | UDCA | β-muri | % Human |
|---|---|---|---|---|---|---|
| T1 | 1.7 | 3.4 | 94.1 | 0 | 0.77 | 75 |
| T2 | 1.5 | 0.9 | 91.7 | 0 | 5.87 | 25 |
| T3 | 2.7 | 2.6 | 85.9 | 0.41 | 8.33 | 10 |
| T4 | 0 | 0 | 91.4 | 0 | 8.6 | 1 |
| T5 | 1.3 | 5.6 | 79.7 | 0.62 | 12.8 | 90 |
| T6 | 0 | 0 | 82.2 | 0 | 17.8 | 20 |
| T7 | 1.1 | 0 | 80.5 | 0 | 18.5 | 1 |
| T8 | 0 | 0 | 73.1 | 0 | 26.9 | 15 |
| T9 | 0.6 | 9.6 | 54.3 | 1.7 | 33.7 | 15 |
| T10 | 0 | 4.0 | 60.7 | 0.65 | 34.7 | 1 |
| C1 | 0 | 5 | 53.9 | 0 | 41.1 | control |
| C2 | 0 | 1.0 | 56.9 | 0 | 42.0 | control |

DCA = deoxycholic acid;
CDCA = chenodeoxycholic acid;
UDCA = ursodeoxycholic acid;
β-muri = beta-muri[3beta-3-H]cholic acid

TABLE 4
Lipid profile of humanized FRG mice

| Animal | Cholesterol | HDL | LDL | Triglycerides |
|---|---|---|---|---|
| C1 | 89 | 46 | 13 | 150 |
| C2 | 72 | 46 | 10 | 138 |
| C3 | 70 | 43 | 8 | 122 |
| T1 | 511 | 68 | 212 | 95 |
| T2 | 166 | 80 | 97 | 94 |
| T3 | 129 | 46 | 40 | 214 |
| T4 | 191 | 74 | 102 | 74 |

Lipids were measured in non-transplanted controls (C1-C3) and highly humanized (>70%) FRG mice (T1-T4).

A pharmacological proteinase inhibitor may be necessary to keep highly repopulated mice viable long term; human complement produced by the donor hepatocytes could injure recipient kidney (see Tateno et al. Am. J. Pathol. 165:901-912, 2004). Therefore, several (n=3) highly repopulated mice were observed for an extended period. These mice did not lose weight while off NTBC for 4 months and their human albumin concentration remained stable. Furthermore, their kidneys were macroscopically and histologically normal at harvest (FIG. 2G).

### Example 5

### Serial Transplantation of Human Hepatocytes

One of the limitations of previously described liver xenorepopulation models is the inability to further expand engrafted human hepatocytes. In order to test the feasibility of serial transplantation in the FRG mouse system, the liver of a highly repopulated primary recipient (−70% human cells) was perfused, and parenchymal hepatocytes were collected using a standard collagenase digestion protocol.

Mice repopulated with human cells were anesthetized and portal vein or inferior vena cava was cannulated with a 24 gauge catheter. The liver was perfused with calcium- and magnesium-free EBSS supplemented with 0.5 mM EGTA and 10 mM HEPES for 5 minutes. The solution was changed to EBSS supplemented with 0.1 mg/ml collagenase XI (sigma) and 0.05 mg/ml DNase I (sigma) for 10 minutes. The liver was gently minced in the second solution and filtered through 70 μm and 40 μm nylon mesh sequentially. After 150×g centrifugation for 5 minutes, the pellet was further washed twice at 50×g for 2 minutes. The number and viability of cells were assessed by the trypan blue exclusion test.

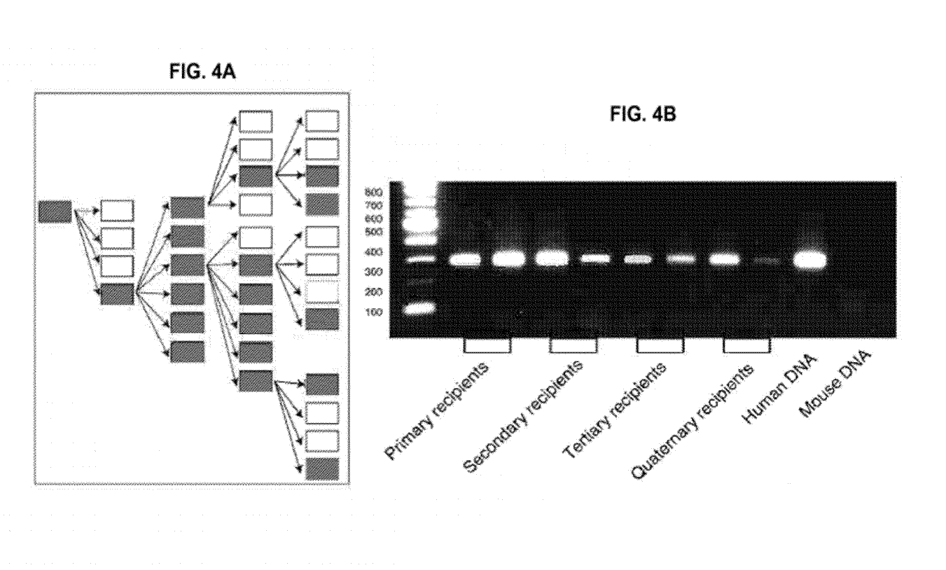

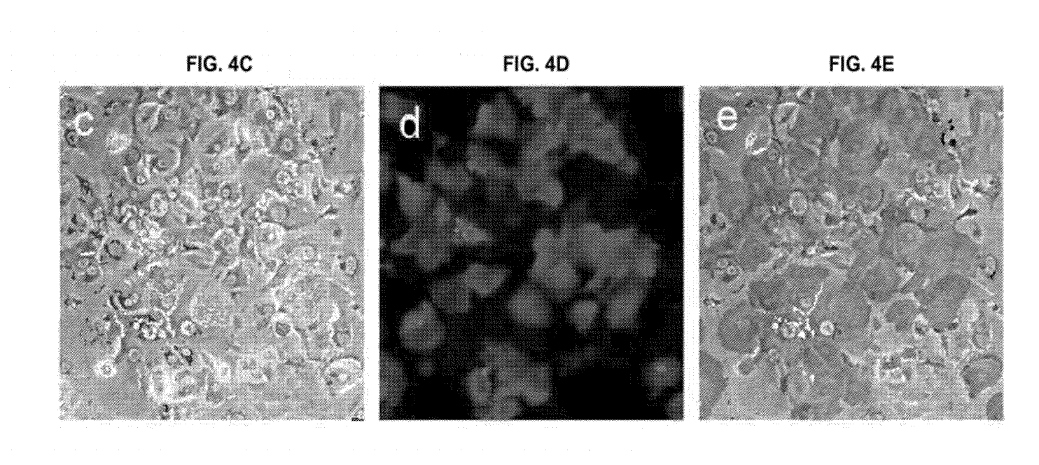

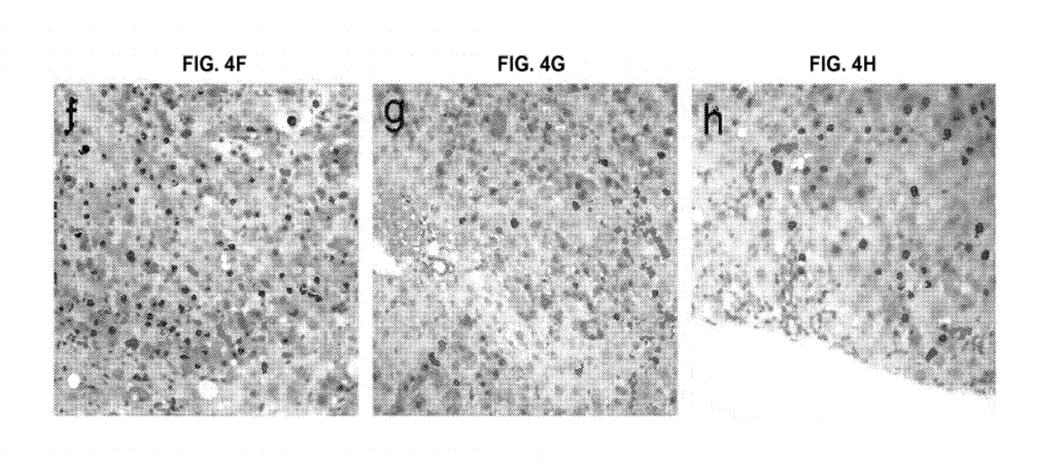

One million viable cells suspended in 100 μl DMEM were injected into recipient spleen via 27 gauge needle. Transplantation of hepatocytes into secondary FRG recipients was performed without separating the Fah-positive human and Fah-negative mouse hepatocytes. In contrast to the cells used for primary engraftment, the viability of human hepatocytes harvested in this fashion was >80%, and they readily attached to collagen-coated culture plates (FIGS. 4C-4E). After engraftment of the secondary recipient, the serial transplantation was continued in a similar fashion into tertiary and quarternary recipients. In each generation, blood human albumin of some, but not all, recipient mice became highly positive (FIG. 4A). The percentage of highly repopulated mice was higher in serial transplant recipients (17/28 compared with 7/43) and the rate of albumin increase was more consistent (FIG. 3E). This may indicate that serial passage of hepatocytes enriches for the most transplantable human hepatocytes or it may simply reflect the higher quality and viability of cells harvested freshly from a donor mouse. Genomic PCR of the liver samples from albumin positive mice showed the presence of human DNA in each generation (FIG. 4B). Liver repopulation by human hepatocytes was also confirmed by fluorescent immunostaining against FAH (FIGS. 4C-4E). Histological examination showed engrafted human hepatocytes were morphologically similar in each generation and were distinctly FAH-positive (FIGS. 4F-4H).

### Example 6

### Hepatocyte Repopulation is not a Result of Cell Fusion

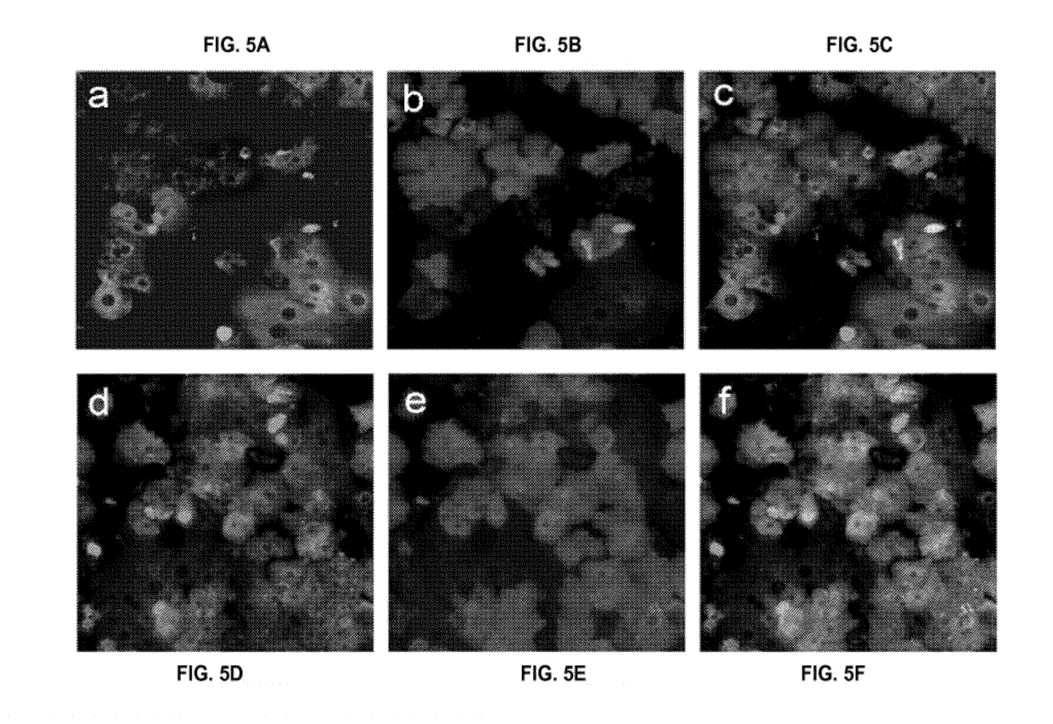

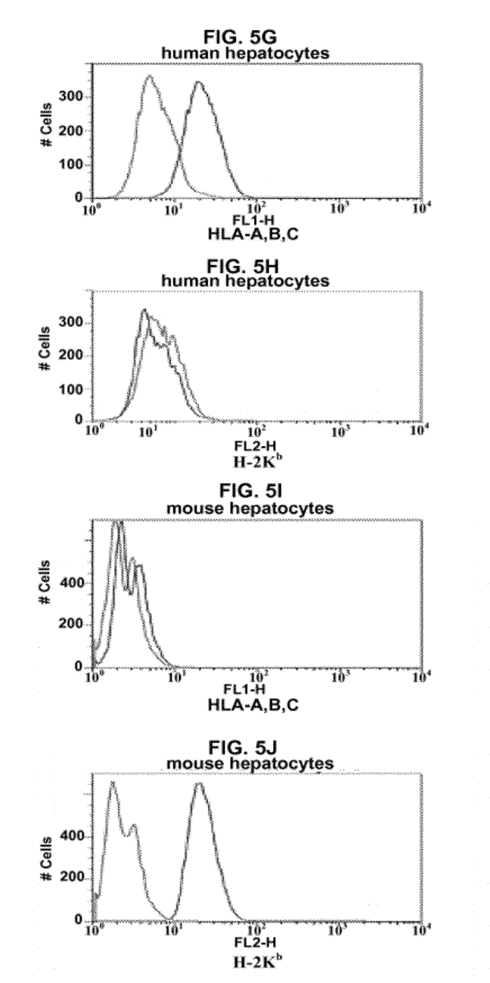

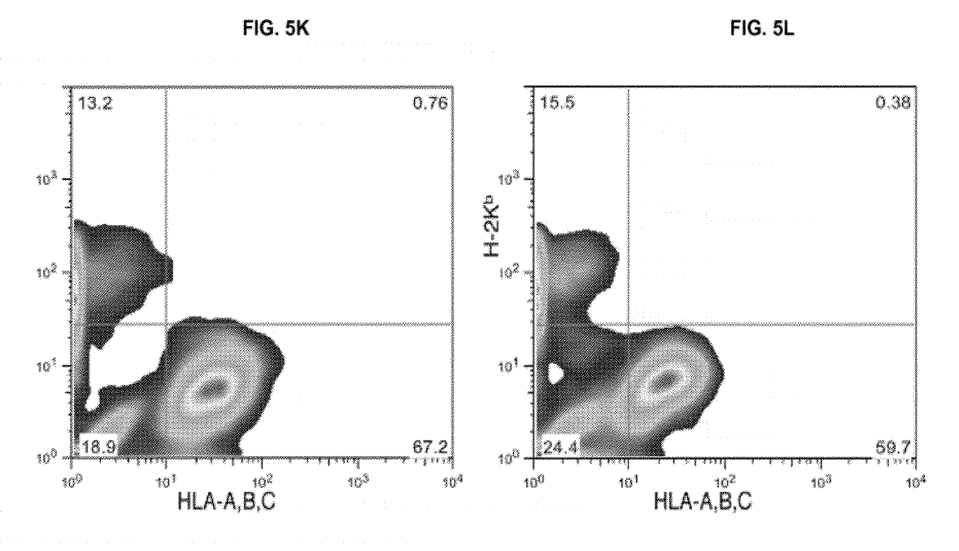

A recent report of liver repopulation with primate cells in urokinase transgenic mice demonstrated that cell fusion could potentially account for apparent “hepatocyte repopulation” (Okamura et al. Histochem. Cell Biol. 125:247-257, 2006). Since uPA-transgenic mice were used in that study, these findings raised the possibility that cell fusion was also the mechanism in other reports of mouse liver humanization. Cell fusion between hematopoietic cells and hepatocytes has also been observed in the Fah-deficient mice (Wang et al. Nature 422:897-901, 2003). Cell fusion between mouse and human cells would greatly diminish the value of humanized mouse livers for pharmaceutical applications. To confirm that the repopulated hepatocytes were truly human in origin, double immunostaining against human- or mouse-specific albumin and FAH was performed. Most (>95%) mouse albumin-positive hepatocytes were indeed negative for FAH and most FAH-positive hepatocytes were negative for mouse albumin (FIGS. 5A-5C). On the other hand, almost all (>90%) human albumin-positive hepatocytes were also FAH-positive, while the remaining hepatocytes were double-negative (FIGS. 5D-5F).

Albumin is a secreted protein and thus cells could appear antibody positive by taking up heterologous albumin from other cells. To further confirm the lack of cell fusion, human and mouse anti-major histocompatibility complex (MHC) antibodies were used for flow cytometry. Each antibody was confirmed to be species-specific (FIGS. 5F-5J). No hepatocytes positive for the surface markers of both species were found in highly repopulated livers (FIG. 5K and FIG. 5L).

Finally, fluorescent in situ hybridization (FISH) was performed with human and mouse whole genome probes on hepatocytes from highly repopulated transplant recipients. Hepatocytes from highly repopulated primary (chimeric mouse #531) and tertiary (chimeric mouse #631) mice were hybridized with either human or mouse total genomic DNA. The percentage of cells positive for the human probe or the murine probe was scored (Table 5). Controls were pure human and mouse hepatocytes or an equal mix of human and mouse hepatocytes. If the human cells found in chimeric livers were the product of cell fusion, many hepatocytes would be double-positive for both human and mouse probes and hence the percentages of cells positive for mouse and human DNA would exceed 100%. Instead, the sum of the percentages closely approximated 100% as it did in the mix of human and murine hepatocytes. Furthermore, human hepatocytes were detected in the spleens of highly repopulated mice (FIG. 2H) despite the fact that the spleen is devoid of murine hepatocytes which could serve as fusion partners for human cells. Thus, double-positive cells (fusion products) could not account for the majority of human cells.

TABLE 5
Detection of human and mouse DNA in repopulated hepatocytes

| | Mouse probe | Human probe | Sum of |
|---|---|---|---|
| | positive (%) | positive (%) | percentages |
| Murine hepatocytes | 87/87 (100) | 0/103 (0) | 100 |
| Human hepatocytes | 0/99 (0) | 107/107 (100) | 100 |
| Mix | 38/101 (38) | 68/115 (59) | 97 |
| Chimeric mouse #531 | 15/100 (15) | 95/111 (86) | 101 |
| Chimeric mouse #631 | 23/87 (26) | 69/94 (73) | 99 |

Taken together, these results indicate that fusion events, if they occurred, were rare and that the majority of repopulating cells were of purely human origin even when serial transplantation was performed. Therefore, human hepatocytes expanded in FRG mice have only human genetic and biochemical properties.

### Example 7

### Functional Characterization of Drug Metabolism in Humanized Mice

The basal expression and induction of human liver specific genes in chimeric mice was examined. Evaluation of testosterone metabolism and ethoxyresorufin-O-deethylase (EROD) activity on cultured hepatocytes was conducted as described by Kostrubsky et al. (Drug Metab. Dispos. 27:887-894, 1999), and Wen et al., (Drug Metab. Dispos. 30:977-984, 2002), respectively. RNA isolation, cDNA synthesis and real-time PCR were conducted as described by Komoroski et al., (Drug Metab. Dispos. 32:512-518, 2004). Primers, obtained from Applied Biosystems, were specific for human CYP1A1 (Hs00153120_m1), CYP1A2 (Hs00167927_m1), CYP3A4 (Hs00430021_m1), CYP3A7 (Hs00426361_a1), CYP2B6 (Hs00167937_g1), CYP2D6 (Hs00164385_a1), Multidrug resistance associated protein MRP2 (Hs00166123_m1), Bile Salt export Pump BSEP, (Hs00184829_m1), CAR (Hs00231959_m1) Albumin (Hs00609411_m1), HNF4α (Hs00230853_m1), Cyclophillin (Hs99999904_m1) and mouse actin (Ma00607939_s1).

Cultures of isolated hepatocytes were established and exposed to prototypical inducers of the cytochrome P450 genes. The results demonstrated that the basal gene expression levels of cytochrome (CYP1A1, CYP1A2, CYP2B6, CYP3A4, CYP3A7), transporter (BSEP, MRP2) and drug conjugating enzymes (UGT1A1) were exactly those found in cultured normal adult human hepatocytes (FIG. 6C, FIG. 7). Furthermore, the pattern of genes induced by compounds such as beta-naphthoflavone (BNF), phenobarbital (PB) and rifampicin (Rif) was as expected from normal human cells. In addition to the mRNA expression levels of human drug metabolism genes, the enzymatic activity of the human CYP1A and 3A family members were measured. Ethoxyresorufin-O-deethylase activity (EROD) is known to be mediated by CYP1A1 and 1A2 in human liver. The results show that EROD activity was specifically and robustly induced by prior exposure to BNF in humanized mouse liver cells (FIG. 6A). Conversely, prior exposure to PB or rifampicin specifically induced the conversion of testosterone to 6-beta-hydroxyltestosterone, a specific measurement of CYP3A4 mediated metabolism (FIG. 6B). Thus, hepatocytes from repopulated FRG livers were indistinguishable from normal human adult hepatocytes in these standard drug metabolism assays.

### Example 8

### Depletion of Macrophages Prior to Hepatocyte Repopulation

Primary engraftment did not occur in 100% of FRG recipient mice, even with urokinase adenovirus pre-administration. It is possible that hepatic macrophages, which are present in normal numbers in FRG mice, limit human cell engraftment by promoting an innate immune response.

To eliminate a potential macrophage-initiated immune response, FRG mice are depleted of macrophages prior to human hepatocyte transplantation. Macrophage depletion can be achieved using any one of a number of methods well known in the art, including chemical depletion (Schiedner et al. Mol. Ther. 7:35-43, 2003) or by using antibodies (McKenzie et al. Blood 106:1259-1261, 2005). Macrophages also can be deleted using clodronate-containing liposomes (van Rijn et al. Blood 102:2522-2531, 2003). Additional compounds and compositions for depleting macrophages are described in U.S. Patent Publication No. 2004-0141967 and PCT Publication No. WO 02/087424. Following macrophage depletion, FRG mice are transplanted, or serially transplanted, with human hepatocytes according to the methods described in the previous Examples herein.

### Example 9

### Engraftment of Human Hepatocytes in F^{pm}RG Mice

FRG mice contain a deletion in exon 5 of the Fah gene (Fah^{Δexon5}). To confirm that human hepatocytes can be engrafted and expanded in any model of Fah deficiency, a mouse strain containing a point mutation in Fah was generated. These mice, called Fah point mutation (Fah^{pm}) mice, have a point mutation in the Fah gene that causes missplicing and loss of exon 7 in the Fah mRNA (Aponte et al., Proc. Natl. Acad. Sci. USA 98:641-645, 2001). No differences in phenotype were detected between Fah^{pm} mice and Fah^{Δexon5} mice.

Fah^{pm} mice were crossed with Rag2^{-/-}/IL1rg^{-/-} mice (as described in Example 1) to produce homozygous Fah^{pm}/Rag2^{-/-}/Il2rg^{-/-} (F^{pm}RG) triple mutant mice. Two cohorts of F^{pm}RG mice were transplanted with human hepatocytes according to the methods described in Example 4. Approximately 24-48 hours prior to hepatocyte transplantation, mice received an intravenous injection (retroorbital) of uPA adenovirus. For comparison, FRG mice were transplanted with human hepatocytes in parallel. Human serum albumin was detected in the peripheral blood of F^{pm}RG mice at highly significant levels (23 μg/ml) two and three months after transplantation. These blood levels of human serum albumin were similar to levels found in FRG mice transplanted at the same time.

These results indicate that F^{pm}RG mice can be repopulated with human hepatocytes to the same extent as FRG mice. Therefore, humanized liver repopulation is not unique to FRG mice with the Fah^{Δexon5} mutation, but can be achieved with any strain of Fah deficient mice.

### Example 10

### Use of an IL-1R Antagonist to Enhance Engraftment of Human Hepatocytes in FRG Mice

Using the human hepatocyte repopulation methods described above (such as in Example 4), it is rare to achieve greater than 50% humanization of mouse livers. Even after transplantation of one million human hepatocytes, serum levels of human albumin are initially undetectable, suggesting that most human hepatocytes are lost during the early stages of repopulation.

It was investigated whether blocking the activation of the IL-1R can abrogate macrophage activation and hence prevent the destruction of transplanted human hepatocytes. Anakinra is a recombinant human IL-1R antagonist (Amgen), which is FDA approved for severe arthritis. Anakinra administration blocks the pathway of inflammatory responses and as demonstrated below, has a positive impact on hepatocyte repopulation.

One million human hepatocytes from the same batch were injected intrasplenically into six FRG mice. In three mice, anakinra (2 mg) was administered intraperitoneally during surgery and then once daily for six additional doses (total dose=14 mg over 7 days). One month after transplantation, blood human albumin was measured by ELISA. Treatment with anakinra remarkably enhanced hepatocyte repopulation. Two of three mice that did not receive anakinra had no detectable human albumin and in one of three mice, anakinra was detected at a concentration of 3 μg/ml. All three anakinra-treated mice had much higher albumin levels (130, 165 and 200 μg/ml).

Additional experiments validated these initial observations. Table 6 below shows the results from three independent tests. Cohorts of genetically identical mice were injected with the same batch of human cells and were either untreated (Con) or treated with anakinra (Ana). Different dosing regimens of anakinra were used and different lengths of administration were tested. High dose anakinra (Hi) was 2 mg/mouse/day and low dose (lo) was 0.4 mg/mouse/day. The drug was injected daily for either 3 days (×3) or 7 days (×7). Blood levels of human albumin were measured by ELISA to ascertain the degree of human repopulation.

TABLE 6
Liver repopulation of FRG mice treated with anakinra

| | Primary | | | | | |
|---|---|---|---|---|---|---|
| | or | Dosing | Elisa #1 | | Elisa #2 | |
| Experiment | Serial | schedule | Con | Ana | Con | Ana |
| #1 | Primary | Anakinra | 20 μg | 4.5 mg | 225 μg | 5.6 mg |
| | | High dose x7 | | 2.0 mg | | 4.8 mg |
| #2 | Serial | Anakinra | 5 μg | 70 μg | 9 μg | 2.6 mg |
| | | Low dose x3 | | 190 μg | | 5.7 mg |
| #3 | Serial | Anakinra | 12 μg | 38 μg-Hi x7 | 53 μg | 159 μg-Hi x7 |
| | | High dose x7 & | 12 μg | 56 μg-Hi x7 | 42 μg | 2000 μg-Hi x7 |
| | | Low dose x3 | | 52 μg-Hi x3 | | 1500 μg-Hi x3 |
| | | | | 60 μg-Hi x3 | | 157 μg-Hi x3 |
| | | | | 8 μg-Lo x7 | | 218 μg-Lo x7 |
| | | | | 32 μg-Lo x7 | | 19 μg-Lo x7 |
| | | | | 8 μg-Lo x3 | | 150 μg-Lo x3 |
| | | | | 24 μg-Lo x3 | | 20 μg-Lo x3 |

Mice treated with anakinra consistently had much higher levels of repopulation. Only mice treated with anakinra reached blood levels in the milligram per milliliter range. On average, anakinra treatment resulted in approximately 100-fold enhanced repopulation. Overall, the high dose (2 mg/day) produced the most consistent results.

These results demonstrate that the percentage of highly humanized FRG mice after transplantation of human hepatocytes is significantly higher with anakinra treatment than without the treatment. For testing of drug metabolism and some virology applications, liver repopulation with human cells must exceed 70%. Thus, increased repopulation efficiency is desirable for a variety of research and therapeutic purposes. In addition, anakinra treatment may enhance the efficiency of human clinical hepatocyte transplantation and make it a more clinically viable procedure.

In view of the many possible embodiments to which the principles of the disclosure may be applied, it should be recognized that the illustrated embodiments are only preferred examples of the disclosure and should not be taken as limiting the scope of the disclosure. Rather, the scope of the disclosure is defined by the following claims. We therefore claim as our invention all that comes within the scope and spirit of these claims.

1. A method of expanding human hepatocytes in vivo, comprising:
transplanting human hepatocytes into an immunodeficient and Fah-deficient mouse, wherein (i) the mouse is further deficient for expression of IL-1R, or (ii) the mouse is administered an IL-1R antagonist; and
allowing the human hepatocytes to expand, thereby expanding the human hepatocytes.

2. The method of claim 1, wherein the human hepatocytes are allowed to expand for at least about 2 weeks, at least about 4 weeks, at least about 8 weeks, at least about 12 weeks, at least about 16 weeks, at least about 20 weeks, at least about 24 weeks, or at least about 28 weeks.

3. The method of claim 1, wherein the immunodeficient and Fah-deficient mouse comprises homozygous disruptions in the Fah gene such that the disruption results in loss of expression of functional FAH protein.

4. The method of claim 3, wherein the disruption comprises an insertion, a deletion or one or more point mutations in the Fah gene.

5. The method of claim 4, wherein the disruption comprises a deletion in the Fah gene.

6-7. (canceled)

8. The method of claim 1, wherein the immunodeficiency of the mouse is due to a genetic alteration, and wherein the immunodeficient and Fah-deficient mouse comprises a genetic alteration selected from the group consisting of recombinase activating gene 1 (Rag1) deficiency, recombinase activating gene 2 (Rag2) deficiency, interleukin-2 receptor gamma chain (Il2rg) deficiency, the SCID mutation, the non-obese diabetic (NOD) genotype, the nude mouse mutation, and combinations thereof.

9. The method of claim 8, wherein the mouse is a Fah^{-/-}/Rag2^{-/-}/Il2rg^{-/-} mouse, a Fah^{-/-}/Rag1^{-/-}/Il2rg^{-/-} mouse, a NOD/Fah^{-/-}/Rag2^{-/-}/Il2rg^{-/-} mouse, or a NOD/Fah^{-/-}/Rag1^{-/-}/Il2rg^{-/-} mouse.

10. The method of claim 9, wherein the mouse is a Fah^{-/-}/Rag2^{-/-}/Il2rg^{-/-} mouse.

11. The method of claim 1, wherein the immunodeficiency is due to immunosuppression, the mouse being administered one or more immunosuppressants to induce the immunodeficiency.

12. (canceled)

13. The method of claim 1, wherein the mouse is deficient for expression of IL-1R.

14. The method of claim 13, wherein the mouse is homozygous for disruptions in the Il1r1 gene, such that the disruption results in loss of expression of functional IL-1R protein.

15. (canceled)

16. The method of claim 14, wherein the mouse is a Fah^{-/-}/Rag2^{-/-}/Il2rg^{-/-}/Il1^{-/-} mouse, a Fah^{-/-}/Rag1^{-/-}/Il2rg^{-/-}/Il1r1^{-/-} mouse, a NOD/Fah^{-/-}/Rag2^{-/-}/Il2rg^{-/-}/Il1r1^{-/-} mouse or a NOD/Fah^{-/-}/Rag1^{-/-}/Il2rg^{-/-}/Il1r1^{-/-} mouse.

17. The method of claim 1, wherein the mouse is administered an IL-1R antagonist.

18. The method of claim 17, wherein the IL-1R antagonist is anakinra.

19. The method of claim 18, wherein anakinra is administered at a dose of about 0.2 to about 6 mg per day, or anakinra is administered for about 5 to about 10 days, or both.

20. (canceled)

21. The method of claim 1, wherein the mouse is administered 2-(2-nitro-4-trifluoro-methyl-benzoyl)-1,3 cyclohexanedione (NTBC) prior to hepatocyte transplantation.

22. The method of claim 21, wherein the NTBC is administered at a dose of about 0.05 mg/kg/day to about 2.0 mg/kg/day.

23. The method of claim 21, wherein the NTBC is administered in the drinking water, in the food or by injection.

24-26. (canceled)

27. The method of claim 1, wherein a vector encoding human urokinase is administered to the mouse prior to transplanting the human hepatocytes.

28-29. (canceled)

30. The method of claim 1, wherein transplanting the human hepatocytes comprises injecting the human hepatocytes into the spleen or portal vein of the mouse.

31. The method of claim 1, wherein the human hepatocytes transplanted into the immunodeficient and Fah-deficient mouse are isolated human hepatocytes.

32. The method of claim 31, wherein the human hepatocytes were isolated from the liver of an organ donor, isolated from a surgical resection or derived from a stem cell, monocyte or amniocyte.

33. (canceled)

34. The method of claim 1, further comprising collecting the human hepatocytes from the mouse.

35. (canceled)

36. The method of claim 34, further comprising expanding the collected human hepatocytes by serial transplantation.

37. The method of claim 1, further comprising collecting a biological sample from the mouse.

38. (canceled)

39. A method for selecting an agent effective for the treatment of a human liver disease, comprising:
(i) administering a candidate agent to an immunodeficient and Fah-deficient mouse transplanted with human hepatocytes, wherein the mouse is further deficient for expression of IL-1R, or the mouse is administered an IL-1R antagonist; and
(ii) assessing the effect of the candidate agent on the liver disease, wherein an improvement in one or more signs or symptoms of the liver disease, indicates the candidate agent is effective for the treatment of the liver disease.

40. The method of claim 39, wherein the human liver disease is infection by a human hepatic pathogen, and the method comprises:
(i) administering a candidate agent to the immunodeficient and Fah-deficient mouse transplanted with human hepatocytes, wherein the mouse is further deficient for expression of IL-1R, or the mouse is administered an IL-1R antagonist, and wherein the transplanted human hepatocytes of the immunodeficient and Fah-deficient mouse are infected with the hepatic pathogen; and
(ii) assessing the effect of the candidate agent on the hepatic infection, wherein a decrease in infectious load of the hepatic pathogen relative to infectious load in the Fah-deficient mouse prior to administration of the candidate agent, indicates the candidate agent is effective for the treatment of infection by the hepatic pathogen.

41. The method of claim 40, wherein the infectious load is determined by measuring titer of the pathogen in a sample obtained from the mouse, by measuring a pathogen-specific antigen in a sample obtained from the mouse, or by measuring a pathogen-specific nucleic acid molecule in a sample obtained from the mouse.

42-45. (canceled)

46. The method of claim 39, wherein the human liver disease is cirrhosis, and the method comprises:
(i) administering a candidate agent to the immunodeficient and Fah-deficient mouse transplanted with human hepatocytes, wherein the mouse is further deficient for expression of IL-1R, or the mouse is administered an IL-1R antagonist, and wherein the immunodeficient and Fah-deficient mouse has been administered a compound that induces the development of cirrhosis in the mouse; and
(ii) assessing the effect of the candidate agent on at least one diagnostic marker of cirrhosis in the immunodeficient and Fah-deficient mouse, wherein the at least one diagnostic marker of cirrhosis is selected from AST, ALT, bilirubin, alkaline phosphatase and albumin, and wherein a decrease in AST, ALT, bilirubin or alkaline phosphatase, or an increase in albumin in the Fah-deficient mouse relative to the Fah-deficient mouse prior to administration of the candidate agent, indicates the candidate agent is effective for the treatment of cirrhosis.

47. The method of claim 39, wherein the human liver disease is hepatocellular carcinoma (HCC), and the method comprises:
(i) administering a candidate agent to the immunodeficient and Fah-deficient mouse transplanted with human hepatocytes, wherein the mouse is further deficient for expression of IL-1R, or the mouse is administered an IL-1R antagonist, and wherein the immunodeficient and Fah-deficient mouse has been administered a compound that induces the development of HCC in the mouse or has been transplanted with malignant hepatocytes; and
(ii) assessing the effect of the candidate agent on HCC in the immunodeficient and Fah-deficient mouse, wherein a decrease in tumor growth or tumor volume in the mouse relative to the mouse prior to administration of the candidate agent, indicates the candidate agent is effective for the treatment of HCC.

48. A method of assessing the effect of an exogenous agent on human hepatocytes in vivo, comprising:
(i) administering the exogenous agent to an immunodeficient and Fah-deficient mouse transplanted with human hepatocytes, wherein the mouse is further deficient for expression of IL-1R, or the mouse is administered an IL-1R antagonist; and
(ii) measuring at least one marker of liver function in the immunodeficient and Fah-deficient mouse, wherein the at least one marker of liver function is selected from AST, ALT, bilirubin, alkaline phosphatase and albumin, and wherein an increase in AST, ALT, bilirubin or alkaline phosphatase, or a decrease in albumin in the mouse, relative to the mouse prior to administration of the exogenous agent, indicates the exogenous agent is toxic.

49. (canceled)
